(19) United States
(12) Patent Application Publication
(10) Pub. No.: US 2007/0092864 A1
(43) Pub. Date: Apr. 26, 2007

(54) Treatment planning methods, devices and systems

(75) Inventors: Joseph Reinhardt, Iowa City, IA (US); John Garber, Westport, CT (US); Juerg Tschirren, Iowa City, IA (US); Milan Sonka, Coralville, IA (US); Geoffrey McLennan, Iowa City, IA (US); Eric Hoffman, Iowa City, IA (US)

Correspondence Address: FULBRIGHT & JAWORSKI L.L.P., 600 CONGRESS AVE., SUITE 2400, AUSTIN, TX, 78701, US

(73) Assignee: The University of Iowa Research Foundation

(21) Appl. No.: 11/542,016

(22) Filed: Oct. 2, 2006

Related U.S. Application Data
(60) Provisional application No. 60/722,176, filed on Sep. 30, 2005.

(51) Int. Cl. C12Q 1/00; G06F 19/00; A61B 5/00

(52) U.S. Cl. 435/4; 600/300; 702/19

(57) ABSTRACT

Treatment planning methods, devices and systems. One of the treatment planning methods includes displaying at least a portion of a set of lungs and one or more potential treatment regions; receiving a selection of a target treatment region from among the one or more potential treatment regions; and displaying one or more locations within the portion that are therapeutically linked to the target treatment region. Other treatment planning methods, devices and systems are included.

### CROSS-REFERENCE(S) TO RELATED APPLICATION(S)

This application claims priority to U.S. Provisional Patent Application Ser. No. 60/722,176, filed Sep. 30, 2005, the entire contents of which are expressly incorporated by reference.

### STATEMENT REGARDING FEDERALLY SPONSORED RESEARCH

This invention was made at least partially with government support under National Institute of Health grant number 1 R43 HL075953-01A1. The government has certain rights in the invention.

### BACKGROUND

1. Field of the Invention

The present methods, devices and systems relate generally to the fields of disease assessment and treatment planning.

2. Description of Related Art

Pulmonary emphysema (Russi and Weder, 2002; Sabanathan et al., 2003) is a chronic, common, debilitating, insidious, yet fatal, progressive disorder of the lungs, that is often related to smoking, but also has a strong familial association (Yim et al., 2004; Bolliger et al., 2004). A 1997 NIH health survey estimated that 3.2 million Americans have been diagnosed with emphysema (Noppen et al., 2004). Emphysema is associated with an expansion of alveolar macrophages in the peripheral lung, a classical marker of chronic inflammation. There is conflicting evidence as to whether emphysema develops because of a simple protease-antiprotease imbalance (Ernst, 2004; Ferson and Chi, 2005). Emphysema presents clinically very late in its course with severe breathlessness, at a time when useful preventative action cannot be undertaken. The disease begins when the patients are in their late 20's, yet clinically is detected in the 40-70 year old age group. In spite of the huge impact of this disease on the community, research regarding epidemiology, or therapeutic strategies, has been slowed because of the lack of a clinical package for objectively assessing the regional characteristics of the lung tissue.

Emphysema is defined as a condition of the lung which is characterized by abnormal, permanent enlargement of air spaces distal to the terminal bronchiole, with destruction of the alveolar walls, and with-out any obvious fibrosis. Destruction in emphysema is defined as non-uniformity in the pattern of respiratory air space enlargement so that the orderly appearance of the acinus and its components is disturbed and may be lost (Henschke et al., 1999). Emphysema has historically been identified and classified according to the macroscopic architecture of the removed, inflated and fixed at a standard pressure, whole lung (Henschke and Yankelevitz, 2000). Such patterns of destruction are clearly a target for objective tissue characterization methodologies using multidetector-row computed tomography (MDCT) imaging, either using histogram methods or more complex measures such as the Adaptive Multiple Feature Method (Aberle et al., 2001; Ellis et al., 2001; Swensen et al., 2005) for lung parenchymal assessment. Certain quantitative approaches have utilized only single first order measures and have largely been limited to mean lung density and assessment of the location of the lower 5th percentile cut-off on the lung density histogram (plotting number of pixels vs. Hounsfield units), and have not been available in an easily usable format.

A widely publicized therapy for pulmonary emphysema is lung volume reduction surgery (LVRS) (Labadi et al., 2005; Conforti and Shure, 2000). Prior to LVRS, therapy was purely supportive care. There is a large national trial (the National Emphysema Treatment Trial, or NETT, the results of which were published in The New England Journal of Medicine in May 2003) that has looked at this modality, with it being unclear as to how to consistently pick the region of the lung to treat using this approach. With LVRS, the obvious emphysematous regions of the lung are removed, while the patient is under anesthesia, using a variety of different techniques and relying on different levels of surgeon skill to decide where and how much lung to remove. MDCT, combined with the identification of the emphysematous regions using histogram analysis, can objectively designate the region of lung that has the most emphysema, and guide the surgeon. Further approaches, using endoscopic lung volume reduction surgery through the airways are currently coming into vogue. Some use aspiration of the affected lung through the airway, and others use some sort of implanted airway valve (Berlin, 2003).

Broadly, lung or pulmonary diseases are the second most common cause of death and morbidity, and there has been a necessary focus on new therapy, including both pharmacologic as well as surgical. A growing number of therapies proceed via endo- and trans-bronchial approaches within the lung, facilitated by rapid advances in optical-based tools and their miniaturization, and by the realization that lung segmental diseases are best treated by segmental or lobar approaches where at all possible. These new therapies include the placement of one-way valves or airway wall fenestrations as an alternative to lung volume reduction surgery for late state emphysema, the placement of stents to resolve airway obstructions, and radiofrequency ablation of airway smooth muscle as a therapy for severe asthma, or of lung tumor nodules.

### SUMMARY

In a broad respect, the invention relates to treatment planning methods and to techniques that can be used during the treatment planning process for diseases such as emphysema and lung cancer, although other diseases and associated tissues are applicable. The invention may take the form of treatment planning methods, devices (such as computer readable media) that may be used as part of the treatment planning process, and systems (such as computer systems) that may be used as part of the treatment planning process.

Some embodiments of the present treatment planning methods comprise displaying at least a portion of a set of lungs and one or more potential treatment regions; receiving a selection of a target treatment region from among the one or more potential treatment regions; and displaying one or more locations within the portion that are therapeutically linked to the target treatment region.

Other embodiments of the present treatment planning methods comprise displaying an airway tree segmented from a volumetric dataset of images; receiving a selection of a portion of the airway tree; and providing a display that includes the portion in straightened form and a dimensional attribute corresponding to a user-selectable location along the portion.

Different aspects of these methods, as well as other treatment planning methods, devices and systems, are described below.

### BRIEF DESCRIPTION OF THE DRAWINGS

The following drawings illustrate by way of example and not limitation. The patent or application file contains at least one drawing executed in color. Copies of this patent or patent application publication with color drawings will be provided by the Office upon request and payment of the necessary fee.

FIGS. 1A-1D: Lung segmentation example according to some embodiments for an image of a normal subject. FIG. 1A—One of the original CT slices. FIG. 1B—After optimal thresholding, yielding the lungs but also yielding the surrounding air. Note that the lungs have interior cavities (due to the blood vessels). FIG. 1C—After filling cavities. FIG. 1D—After smoothing the region around the mediastinum.

FIG. 1E: Lung segmentation flow chart.

FIGS. 1F-1G: Lung smoothing. FIG. 1F—Vessel indentation. FIG. 1G—Airway protrusions.

FIGS. 1H-1I: Lobe segmentation. FIG. 1H—Sagittal slice from right lung showing oblique and horizontal fissures. FIG. 11—Sagittal slice from left lung showing oblique fissure.

FIG. 1J: Lobe segmentation flow chart.

FIG. 1K: Basins and merges for a 1-D gray level topography.

FIG. 1L: Anatomically labeled airway tree.

FIGS. 1M—1N: Watershed segmentation result for (1M) right lung sagittal slice, (1N) left lung sagittal slice.

FIGS. 1O-1P: (1O) Fissure ROI, (1P) Angle of rotation.

FIG. 1Q-1R: Sagittal plane from bounding box showing (1Q) Rotated ROI, (1R) Cost function.

FIG. 1S: Watershed segmentation result for a right lung sagittal slice.

FIG. 1T: Horizontal fissure ROI.

FIG. 1U-1V: Sagittal plane from bounding box showing (1U) ROI, (1V) Cost function.

FIGS. 2A-2C: 2D example showing separation of lung into core and rind. Morphological erosion is used to peel away boundary pixels. FIG. 2A shows the original segmented lung image; FIG. 2B shows the core and rind after peeling away 15 pixels around the boundary; FIG. 2C shows the core and rind after peeling away 30 pixels around the boundary.

FIGS. 3A and 3B: Example of segmentation and skeletonization applied to an airway tree phantom. FIG. 3B is a closeup of the skeleton.

FIGS. 4A and 4B: Example of anatomical labeling. FIG. 4A shows the 33 labels that are automatically assigned. FIG. 4B shows a detail view of a labeling result, and shows two false branches at the carina. The labeling result is correct despite the false branches.

FIG. 5A: 3D rendering of a segmented vascular tree, showing the position of the fissures (highlighted by arrows) as a separation in the otherwise dense tree.

FIG. 5B: Sagittal slice of a dataset showing segmented lobes in the right lung.

FIG. 6: Log-log plot of the histogram of air-cluster sizes, determined by an embodiment of the low attenuation cluster (LAC) analysis discussed below.

FIG. 7: The “Start” screen for the embodiment of Pulmonary Workstation described below.

FIG. 8: The “Load Patient” dialog box triggered by choosing the “Review/Analyze Screen” option on the “Start” screen shown in FIG. 7.

FIG. 9: The folder browsing window triggered when the “New Patient” button shown in the FIG. 8 box is selected.

FIG. 10: The “Select Scan” window allowing a user to browse for files within a given folder selected at the folder browsing window shown in FIG. 9.

FIG. 11: The “Edit Patient Database” window triggered when the “Administer Patients” button on the Start screen is selected.

FIG. 12: The “Analyze Study” screen for the embodiment of Pulmonary Workstation described below.

FIG. 13: One version of the “Bronchial Tree” screen for the embodiment of Pulmonary Workstation described below.

FIG. 14A: An example of a bronchial tree (from the “Bronchial Tree” screen) in which a certain airway path has been selected consistent with the embodiment of Pulmonary Workstation described below.

FIG. 14B: An example of the “Bronchial Tree” screen in which the bronchial tree has been enlarged using the zoom function, an airway segment has been selected, and the user is considering whether to rename it.

FIG. 15: An example of the “Lumen View” screen for the embodiment of Pulmonary Workstation described below, where different features are labeled/described.

FIG. 16: Another example of the “Lumen View” screen for the embodiment of Pulmonary Workstation described below. In this example, the position plane is close to a distal end of the airway tree.

FIG. 17: An example of a spreadsheet showing measurement data determined using the embodiment of Pulmonary Workstation described below.

FIG. 18: An example starting screen that may be generated using the embodiment of Pathfinder described below.

FIG. 19: An example display generated using the embodiment of Pathfinder described below, in which a user has selected a target sphere (representing an low attenuation cluster indicative of emphysematic lung tissue) and that path to that target sphere has been highlighted.

FIG. 20: An example display generated using the embodiment of Pathfinder described below, in which an airway segment has been selected and the tissue fed by it does not include the target tissue (note the red line is drawn to a sphere that is not highlighted).

FIG. 21: An example display generated using the embodiment of Pathfinder described below, in which the bronchial tree and displayed LACs have been rotated using a click-and-drag function.

FIG. 22: Values of slope α in log(count)=α·log(size)+β for low attenuation cluster (LAC) analysis at −990 Houndsfield units (HU). Mean values are μ_{α emphysema}=−1.234 and μ_{α normal}=−1.599. A Welch Two Sample t-test rejects the null hypothesis of μ_{α emphysema}=μ_{α normal} with p=1.65 10⁻¹¹.

FIG. 23: Comparison of observer-defined and computer-segmented inner and outer airway wall borders. The top row comprises expert-traced borders. The bottom row comprises 3D surface obtained using the segmentation technique described below (characterizable as a “double-surface” segmentation technique).

FIGS. 24A-24C: Results of valve experiment. FIG. 24A is a screen shot from the use of Pathfinder (described below), showing a CT slice overlaid on the segmented airway tree and simulated emphysema regions (red spheres). FIG. 24B depicts the same segmented airway tree shown in FIG. 24A without the CT slice and where the pathway (in red) to one of the emphysema regions is more clearly visible. FIG. 24C is a CT scan showing the inserted valves highlighted by arrows.

FIG. 25: Results of ventilation and perfusion study from one sheep before and after inserting two one-way valves at locations determined using Pathfinder (described below). The left-most upper and lower scans are before and after CT images. The middle upper and lower images show ventilation in milliliters/minute within the region of the lungs depicted in the left-most scans before and after valve placement. The right upper and lower images show blood flow (perfusion) in milliliters/minute to the region of the lungs depicted in the left-most scans before and after valve placement.

### DESCRIPTION OF ILLUSTRATIVE EMBODIMENTS

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. Thus, a method “comprising” displaying at least a portion of a set of lungs and one or more potential treatment regions; receiving a selection of a target treatment region from among the one or more potential treatment regions; and displaying one or more locations within the portion that are therapeutically linked to the target treatment region is a method that includes at least these recited steps, but is not limited to only possessing these recited steps.

Similarly, a computer readable medium “comprising” machine readable instructions for performing certain steps is a computer readable medium that has machine readable instructions for implementing at least the recited steps, but also covers media having machine readable instructions for implementing additional, unrecited steps.

The terms “a” and “an” are defined as one or more than one, unless this application expressly requires otherwise. The term “another” is defined as at least a second or more.

### Lung Segmentation

In some embodiments, the lungs and the bronchial tree may comprise a region or volume of interest. In such embodiments, lung segmentation may be performed with the goal of separating the voxels corresponding to lung tissue from the voxels corresponding to the surrounding anatomy. The dataset in which the voxels appear may be created using any suitable imaging modality, such as MDCT, computed tomography (CT), magnetic resonance imaging (MRI), positron emission tomography (PET), confocal microscopy, or volumetric ultrasound, to name a few. The dataset may be characterized as a volumetric dataset of images, which is a dataset comprised of a series of cross-sectional images of the region of interest (e.g., the mediastinum). Following an approach that has been used for segmentation in normal subjects (Hu et al., 2001), a combination of optimal thresholding, region connectivity and topology analysis may be used to identify the lungs. After segmentation, the left and right lungs may be separated using dynamic programming.

Optimal thresholding is an automatic threshold selection method that allows the user to accommodate the normal variations in tissue density expected across a population of subjects. The segmentation threshold may be selected through an iterative procedure. For example, let Tⁱ be the segmentation threshold at step i. To choose a new segmentation threshold, apply Tⁱ to the image to separate the voxels into body and non-body voxels. Let μ_{b} and μₙ be the mean gray level of the body voxels and non-body voxels after segmentation with threshold Tⁱ. Then the new threshold for step i+1 is: Tⁱ⁺¹=½=(μ_{b}+μₙ). This iterative threshold update procedure may be repeated until there is substantially no change in the threshold, e.g., Tⁱ⁺¹=Tⁱ. The initial threshold T⁰ may be selected based on the CT number for pure air (−1000 HU) and the CT number for voxels within the chest wall/body (>0 HU).

After applying the optimal threshold, 3D connected components labeling may be used to identify the lung voxels. The background air may be eliminated by deleting regions that are connected to the border of the image. Small, disconnected regions may be discarded if the region volume is too small. To identify the lungs, only the two largest components in the volume may be retained, with the additional constraint that each component must be larger than a predetermined minimum volume. The high-density blood-filled vessels in the lung may be labeled as body voxels during the optimal thresholding step. As a result, the 3D lung regions will contain unwanted interior cavities. Topological analysis, similar to that used in (Hu et al., 2001; Reinhardt and Higgins, 1998), may be used to fill the lung regions and eliminate the interior cavities.

The trachea and left and right mainstem bronchi may be identified in the original gray level image data using a closed-space dilation with a unit radius kernel (Masutani et al., 1996). This procedure is equivalent to directed slice-by-slice region growing. To initialize the closed-spaced dilation, the location of the trachea may be automatically identified by searching for the large, circular, air-filled region near the center of the first few slices in the data set. Regions in the current slice provide potential seed point positions for the next slice. The slice-by-slice growing procedure may be stopped when the size of the region on a new slice increases dramatically, indicating that the airways have merged into the low-density lung tissue.

When viewed on transverse CT slices, the anterior and posterior junctions between the left and right lungs may be very thin with weak contrast. Grayscale thresholding may not separate the left and right lungs near these junctions. The left and right lungs may be separated using a 2D dynamic programming technique applied on the transverse slices (Hu et al., 2001; Brown et al., 1997), driven by a graph containing weights proportional to pixel gray level. The maximum cost path corresponds to the junction line position.

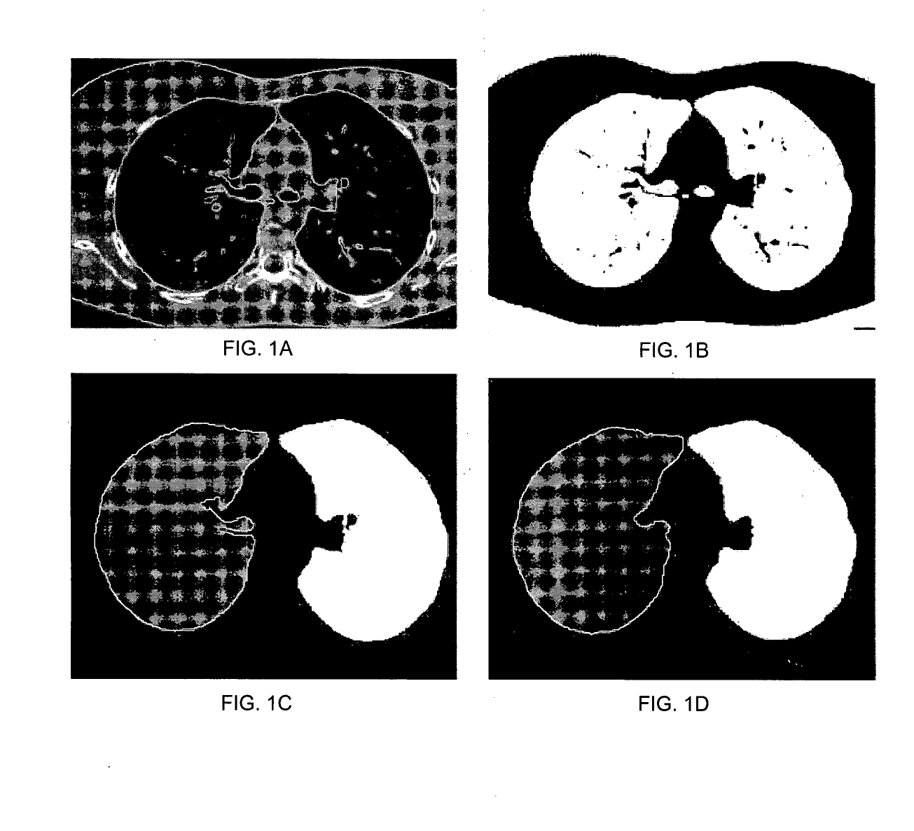

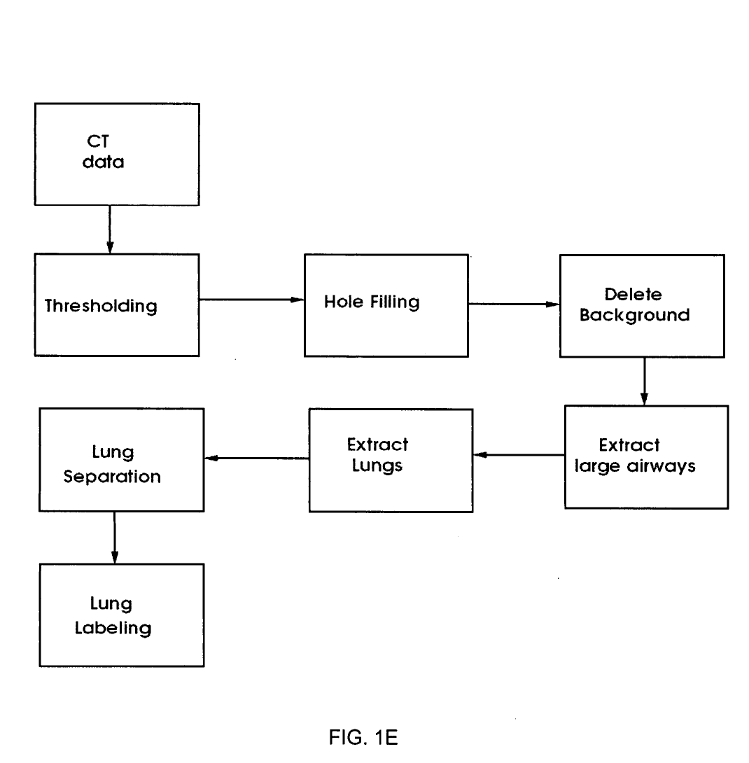

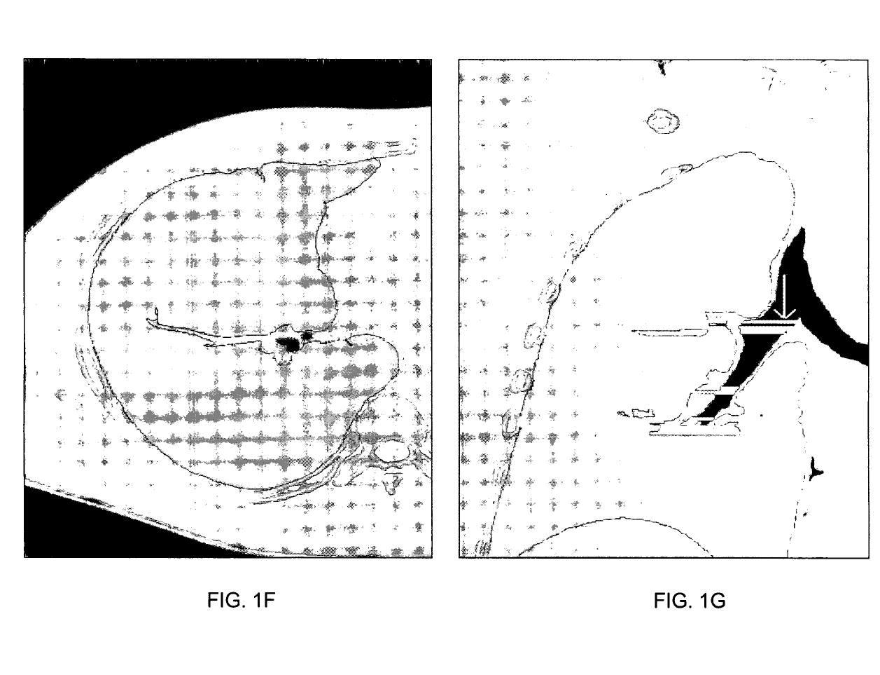

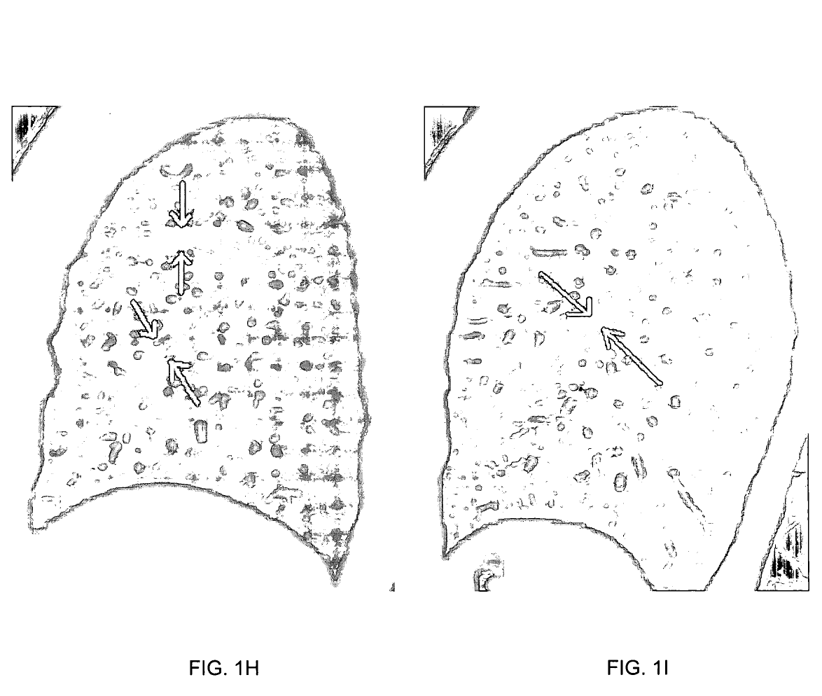

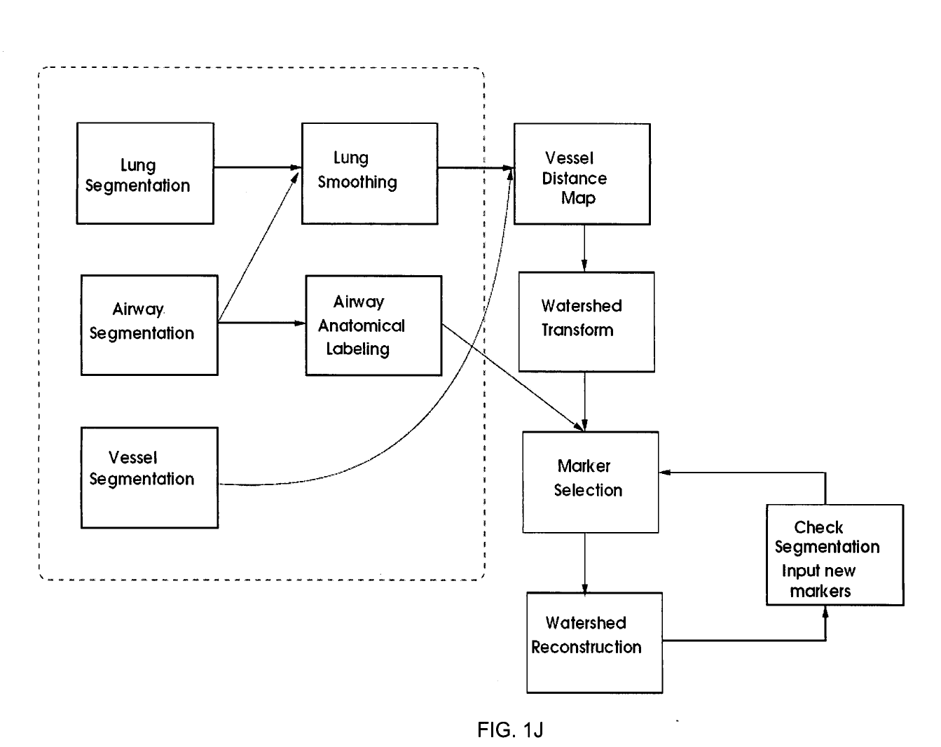

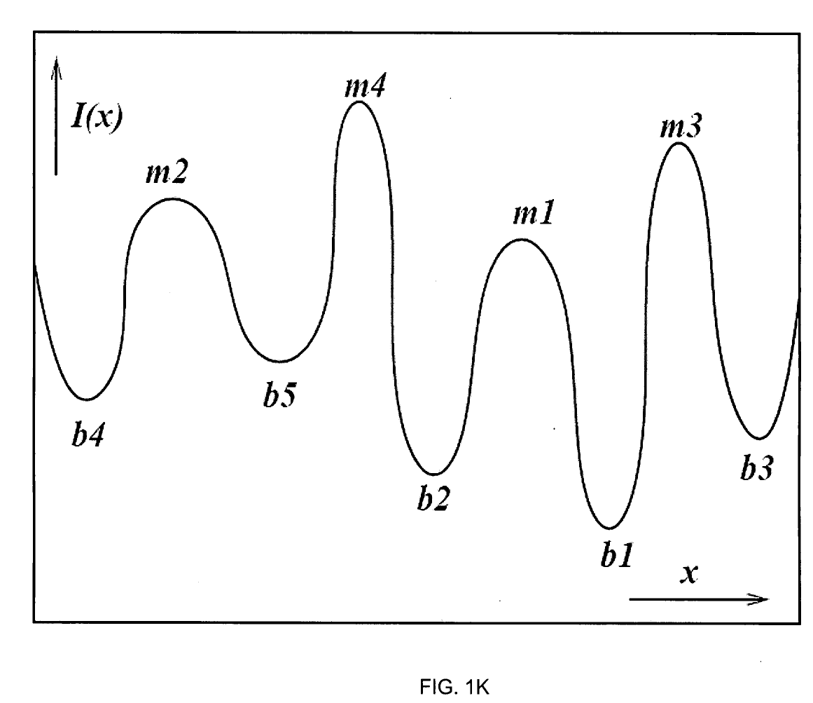

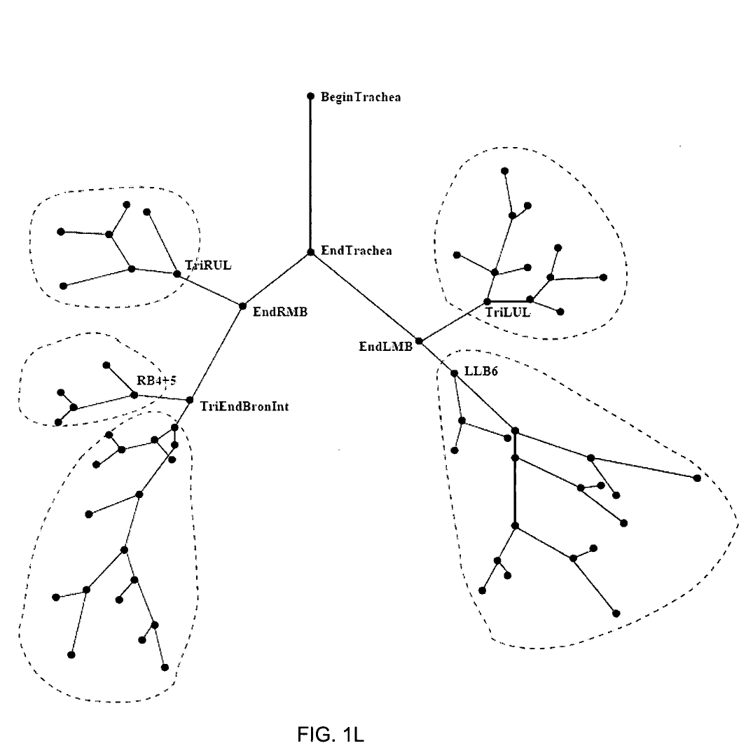

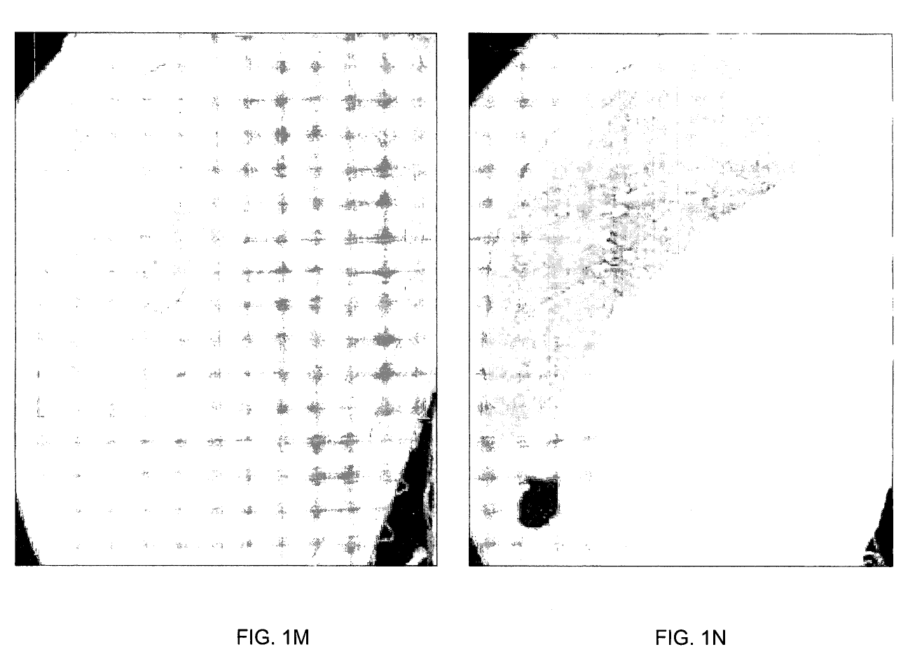

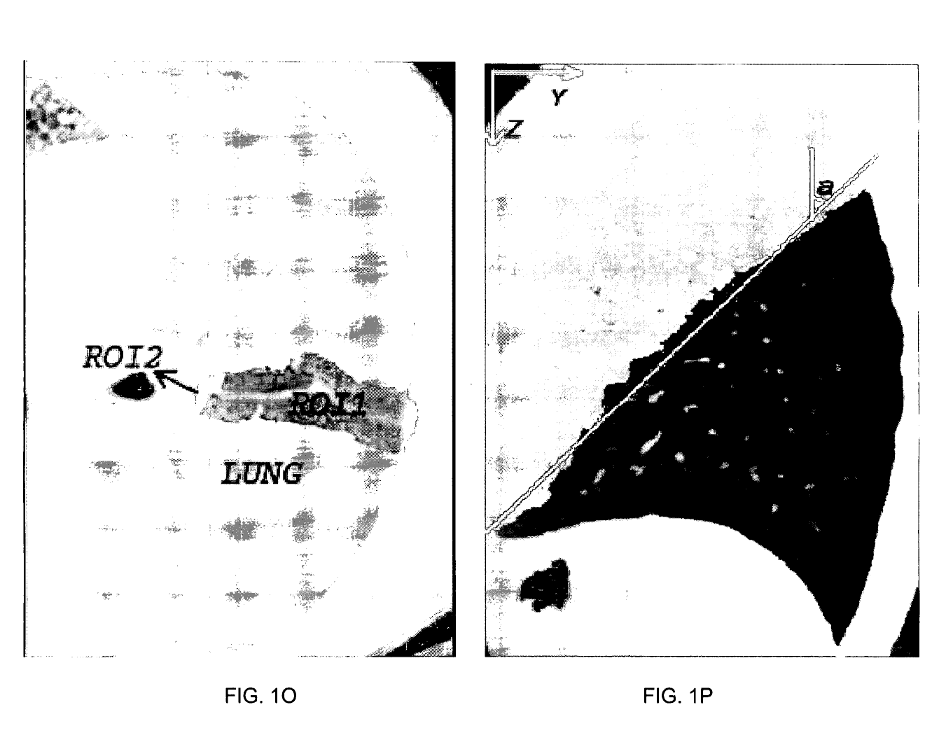

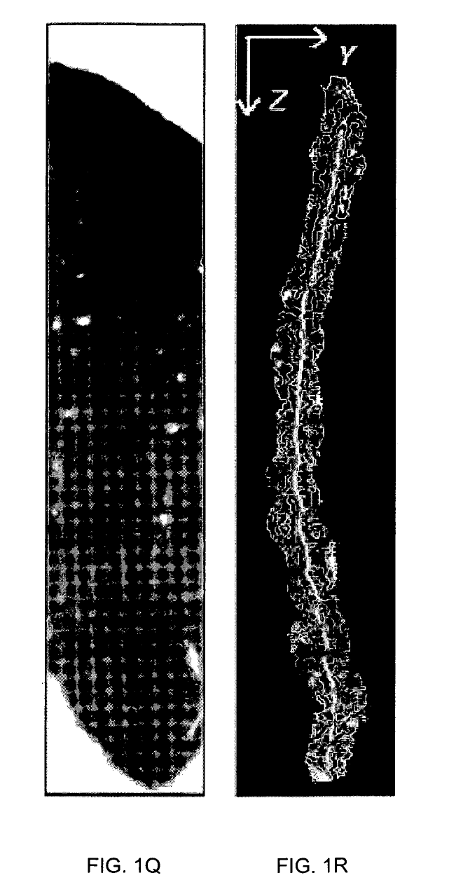

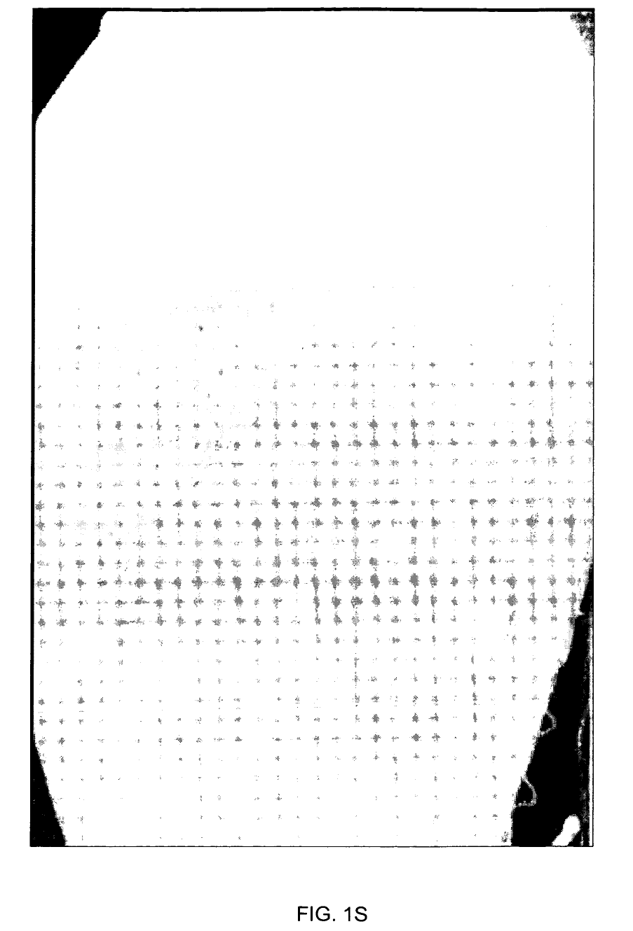

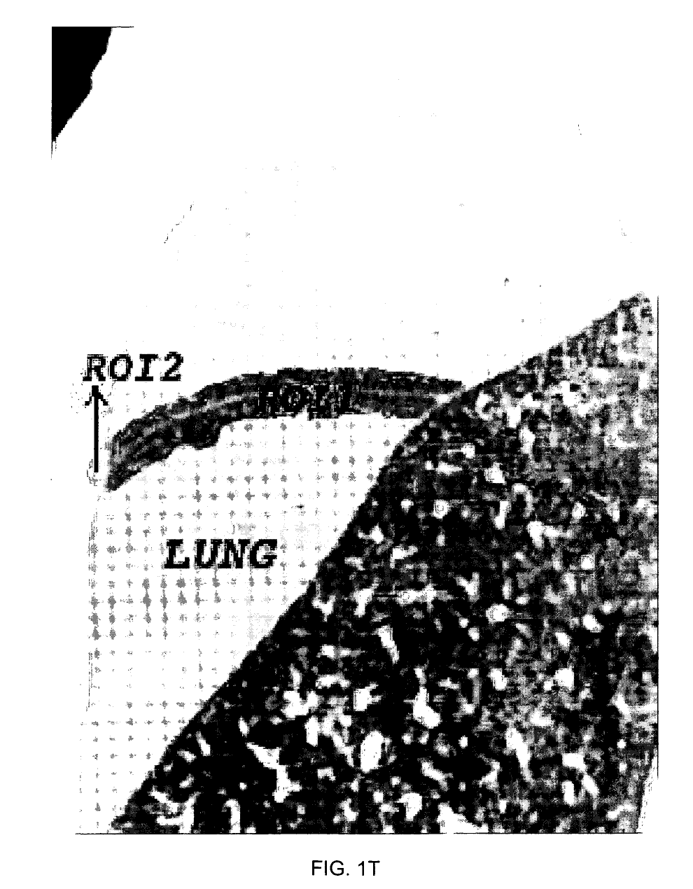

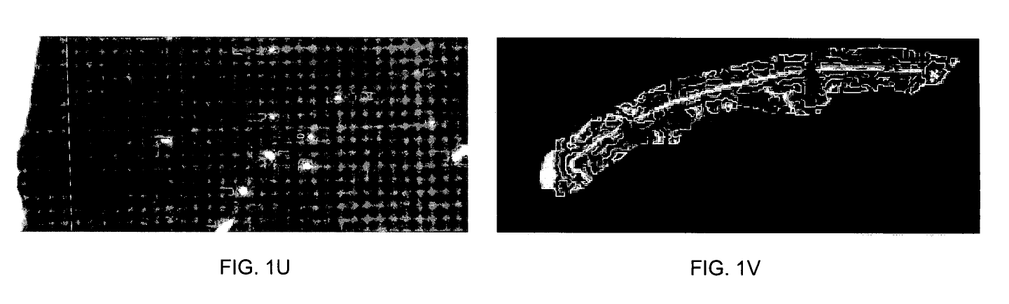

The lung regions near the mediastinum contain the radio-dense pulmonary arteries and veins. By the gray level processing described above, these vessels may be excluded from the lung regions. But when manually tracing the lung contours, a manual analyst may “cut” across the large pulmonary vessels and group them with the lung regions, yielding a smooth lung contour. A similar situation occurs as the mainstem bronchi merge into the lungs. A lung boundary shape smoothing step may be included to give a smoothed lung boundary that more closely-mimics the borders defined by some manual analysts. The smoothing step may use operations from binary mathematical morphology to address three shape-related issues: boundary indentations caused by the large pulmonary vessels, large boundary bulges caused by the left and right mainstem bronchi merging into the lungs, and small boundary bulges caused by small airways near the lung borders. Results of the initial grayscale segmentation, left and right lung separation, and smoothing steps are shown for a normal subject in FIGS. 1A-1D.

The lung segmentation technique described below is one manner of carrying out lung segmentation consistent with some embodiments of the present methods, devices and systems:

1.1 Lung Segmentation Introduction

Lung segmentation is the automatic identification of the left and right lungs from chest CT scans.

1.1.1 Inputs

16-bit X-ray CT image object.

1.1.2 Outputs

8-bit volume with separate labels for right and left lung.

1.1.3 Orientation

The lungs are assumed to be oriented such that, in the memory representation, X increases from the right of the patient to the left, Y increases from the front of the patient to the back, and Z increases from the head to the feet.

1.2 Algorithm

The method described below is referred to by (Hu et al., 2001). It comprises the following principal steps:
Gray level thresholding to extract the lung regions
Connected component analysis of thresholded structures
Separation of the right and left lungs using dynamic programming
   A lung segmentation flow chart is depicted in FIG. 1E.
   1.2.1 Thresholding

For thresholding the CT data, an optimal threshold selection procedure is used (Sonka et al., 1999). Optimal thresholding is an automatic threshold selection method that allows us to accommodate the small variations in tissue density expected across a population of subjects. For this step, we assume that the image volume contains only two types of voxels: 1) voxels within the very dense body and chest wall structures (the body voxels); and 2) low-density voxels in the lungs or in the air surrounding the body of the subject (the non-body voxels). We will use optimal thresholding to select a segmentation threshold to separate the body from the non-body voxels, and then identify the lungs as the low-density cavities inside of the body.

The segmentation threshold is selected through an iterative procedure. Let Tⁱ be the segmentation threshold at step i. To choose a new segmentation threshold, we apply Tⁱ to the image to separate the voxels into body and non-body voxels. In this case all voxels lesser than Tⁱ are assumed to be non-body. Let μ_{b} and μₙ be the mean gray-level of the body voxels and non-body voxels after segmentation with threshold Tⁱ. Then the new threshold for step i+1 is:

$${T}^{ i + 1} = \frac{{μ}_{b} + {μ}_{n}}{2}$$

This iterative threshold update procedure is repeated until there is no change in the threshold, i.e., Tⁱ⁺¹−Tⁱ<tolerance. We set a tolerance value equal to 1. The algorithm is initialized by computing μₙ from the eight corner voxels in the 3-D volume, and μ_{b} from the remaining voxels.
1.2.2 Connectivity and Topological Analysis

After applying the optimal threshold, the non-body voxels (foreground) will correspond to the air surrounding the body, the lungs, and other low-density regions within the image volume (i.e., gas in the bowel). The following steps are then used to retain only the lungs as the solitary binary objects.

Filling of Holes

The high-density vessels in the lung will be labeled as body voxels during the optimal thresholding step. As a result, the 3-D lung regions will contain unwanted interior cavities. Topological analysis is used to fill the lung regions and eliminate the interior cavities:
Invert binary image so that background becomes foreground and vice versa.
Select largest foreground object and delete remaining objects
Invert binary image back.

The above method ensures that all the small 3-D holes inside the lung are filled. A 2-D hole filling procedure is not used because it sometimes leads to filling in of the region near the heart on some lungs, causing problems during lung separation later.

Delete Background

We need to delete the background air to ensure that the lungs are the biggest objects left. For every transverse slice we do seeded region growing from the four comers of the slice, and set all connected voxels to the background label.

Segmentation of the Large Airways

The trachea and left and right mainstem bronchi are identified in the original gray-level image data using a closed-space dilation (Masutani et al., 1996):
For a fixed number of transverse slices from the top of the dataset, search for most circular foreground object near the middle of the dataset.
The Circularity is defined by the ratio object_width/object_length. Only those objects for which the circularity is greater than 0.4 are considered. The object with maximum circularity is picked as trachea.
Further, select only those objects whose area lies between some pre-set area limits, 0.0005*Slicevolume to 0.009*Slicevolume.
Once a trachea object is found pick any of its voxels as trachea seed.
Starting with slice below trachea seed:
   Select seed from intersection of foreground object on current slice and trachea object on slice above
   Perform seeded region growing on current slice with trachea label
   If area of region grow object<(maximum trachea area on previous slice)*3.0, accept result and keep on growing
   else relabel object as foreground
   Do until no trachea candidates found on a given slice
Temporarily delete all trachea points from foreground and store.
   Extract Lungs

After the closed-space dilation, we use 3-D connected component analysis to extract all components larger than Dataset.volume*LUNG_VOLUME_RATIO—the ratio pre-defined as 0.011. On most datasets the 2 lungs are connected and we have to separate the right and left lungs. Before right and left lung separation, we process every transverse slice to delete all foreground objects smaller than 0.1*(Size of biggest object). During this step we also detect those slices on which the lungs are connected, by checking the area of the lungs. If there is one big connected object larger than 0.038*Slice_volume, the slice is put on the stack of connected slices.

Filling of Holes—2D

At this step a copy of the lung mask image is made, LungCopy. A 2-D hole filling operation is done on the lung mask image. For each transverse slice all the holes in the foreground are filled in, similar to the hole filling procedure described above, in 3-D. This step is undertaken to take care of the cases where the region near the heart appears as a hole.

1.2.3 Left and Right Lung Separation

For the cases in which the lungs are connected, separation is performed using a 2 step process. First the approximate joining region is found using morphological operations. Then dynamic programming is performed slice by slice to actually separate the lungs.

Finding Connection Region

For all slices determined to have connected lungs, as described above, the connection region is determined by the following method:
do
   erode lung object by unit disk shaped structuring element, B4. while there is only one object with size>1/2*MinLungVolume
if number of objects==2% assume lungs separated
   erode twice to get clearer joining region
   do constrained dilation of the connected lungs
   subtract dilated result from original lungs to get connection regions
   check all joining regions for true joining regions (A true joining region is one, which when added back to the original slice, causes it to become one connected object)
else
   failed to find joining region

MinLungVolume=0.01*Slice_volume. The unit disk structuring element, B₄, is shaped like the 4-connected neighborhood of a voxel.

Constrained Dilation

Constrained dilation is a morphological operation defined by the following algorithm:
Inputs→Eroded image and original image
Output→Constrained dilated image
Label the objects on eroded image with separate labels
Put each boundary voxel on eroded image in a stack
do
   pop voxel from stack
   for each 8 connected neighbor of voxel
      if original image label for neighbor is foreground
         if neighbor has same label or background label on eroded image
         Output image neighbor is assigned foreground label
         neighbor on eroded image is assigned same label
         push neighbor onto stack
while stack is not empty
   Separation of Connected Slices
for each connected slice
   if joining region already found on previous slice
   separate lungs using dynamic programming in joining region
   if number of segments!=2% separation failure
      find joining region on current slice
      if number of joining regions>2 or number of joining regions==0 separation failure, add slice number to stack for failed separation
      else
         separate lungs using dynamic programming in joining region
   else
      find connected region on current slice
      if number of joining regions>2 or number of joining regions==0
         separation failure, add slice number to stack for failed separation
      else
         separate lungs using dynamic programming in joining region
   if length of separating line found by dynamic programming>length threshold % Region near heart has been filled wrongly as a hole causing longer junction
   Store slices with hole filling problem
      length threshold=30

Dynamic programming is applied to find the maximum cost path through a graph with weights equal to pixel gray-level, similar to the method described in (Brown et al., 1997). The maximum cost path corresponds to the junction line position.

For slices with hole filling problem, do the following steps:
for each problem slice
   find intersection of current mask and LungCopy(*)
   fill in the 2-D holes in all transverse slices
      *Section: Filling of Holes—2D
      1.2.4 Lung Labeling

After separation the lungs are separated on all transverse slices, but they might still be connected in the 3-D connectivity sense. The labeling is done in a slice by slice approach. We use the fact that on transverse slices, the right lung is in the left half of the dataset and vice-versa. First, a seed point is detected for each lung, in the following manner:

```
set counter = 0
for the middle transverse slice
  scan along Y followed by X, from top left to bottom right
    if voxel = = foreground
      counter++
      if counter = = 1
        do 2-D region growing with right lung label
        save right center point
      if counter = = 2
        do 2-D region growing with left lung label
        save left center point
        break
while counter ! =2
```

repeat above procedure for slices above and below middle slice
(center point—mean x and y coordinates for a region)

After the initial seeds have been found, the labels are propagated slice by slice. The basic assumption is that the center points for a lung on one slice will lie inside the corresponding lung on an adjacent slice. The algorithm can be described as follows:
for slices above and below initial slice
   if right center point is in foreground
      do 2-D region growing with right lung label
      update right center point
   else
      search within a window for new right lung seed point
      do 2-D region growing with right lung label
      update right center point
for slices above and below initial slice
   if left center point is in foreground
      do 2-D region growing with left lung label
      update left center point
   else
      search within a window for new left lung seed point
      do 2-D region growing with left lung label
      update left center point

For the right lung, the search window is of size XDim/5 to the left of current center point X coordinate. For the left lung, the search window of size XDim/5 to the right of current center point X coordinate. XDim is the size of the image object in X. Finally, any unlabelled voxels left are labeled according to the labels of connected voxels.

The lung smoothing technique described below is one manner of carrying out lung smoothing consistent with some embodiments of the present methods, devices and systems:

2.1 Lung Smoothing Introduction

Lung smoothing is a post processing step after lung segmentation, to smooth the contours of the lungs, especially on the inner surface near the mediastinum.

2.1.1 Inputs

8-bit lung segmentation image object, 8-bit airway tree image object.

2.1.2 Outputs

8-bit volume with separate labels for right and left lung.

2.1.3 Orientation

The lungs are assumed to be oriented such that, in the memory representation, X increases from the right of the patient to the left, Y increases from the front of the patient to the back, and Z increases from the head to the feet.

2.2 Algorithm

The unsmoothness in the contour of the lungs is caused due to 2 principal reasons.
1. The high density vessel segment near the lung borders are excluded from the lungmask and appear as indentations. See FIG. 1F.
2. The low density airway tree segments are connected to the lung masks on some transverse slices, appearing as protrusions from the lung surface. See FIG. 1G.
   Each of these issues is addressed separately.
   2.2.1 Separating the Airways
First we dilate the airway tree mask with the structuring element B₄. This is done because the airway tree segmentation represents only the lumen, and we want to include the airway wall also.
Compute the set difference between the lung mask and the airway mask.
We do a morphological opening with B₄, to clean up some small protruding segments left after the airway tree removal.
For all transverse slices, we delete objects smaller than 0.1*(Size of biggest object).
   The unit disk structuring element, B₄, is shaped like the 4-connected neighborhood of a voxel.
   2.2.2 Morphological Smoothing

After the first step, we are left with a lung mask with indentations on the lung surface corresponding to the vessels.
Find bounding box for each lung, left and right.
Within bounding box, do a morphological closing. The structuring element size was experimentally determined, and we use a disk of radius 5 voxels in the XY plane.
The removal of the airways causes holes inside the lung regions. A 2-D hole filling operation is performed on all transverse slices. This method is the same as used in lung segmentation.

### Identification of Rind and Core

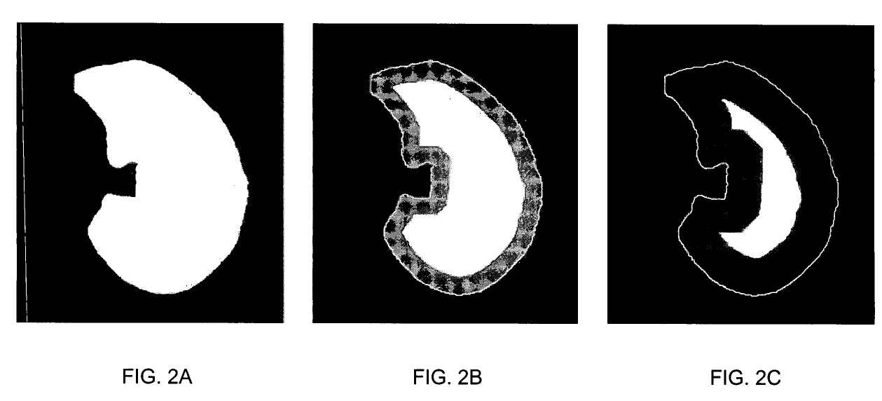

In some embodiments, once the lungs have been identified, the lung regions may be divided into surgically-accessible and surgically-inaccessible subregions using 3D “rind” and “core” algorithms, respectively (Guo et al., 2002). The core and rind may be useful for surgical planning for an intervention such as LVRS. Operations from mathematical morphology may be used to “peel” away a prescribed thickness of boundary pixels to identify the rind, leaving only the core. FIGS. 2A-2C illustrate this method in 2D, however, in an actual implementation this identification is performed in 3D.

### Airway Tree Segmentation and Skeletonization

In some embodiments, airway segmentation takes advantage of the relatively high contrast in, for example, CT images between the center of an airway and the airway wall. A seeded region growing may be used, starting from an automatically-identified seedpoint within the trachea, similar to the method used in (Hu et al., 2001). New voxels may be added to the region if they have a similar X-ray density as a neighbor voxel that already belongs to the region. The tree may be segmented multiple times using more and more aggressive values for measuring the similarity between neighboring voxels. At some point, the segmentation may start to “leak” into the surrounding lung tissue. This may be detected by measuring the volume growth between iterations. A sudden big increase in volume indicates a leak and the result from the previous iteration step may be taken as end-result. A breadth-first search (Silvela and Portillo, 2001) may be used, which allows a fast and memory-friendly implementation. After airway segmentation, a binary subvolume may be formed that represents the extracted airway tree.

In some embodiments, the segmented airway tree may be skeletonized to identify the three-dimensional centerlines of individual branches and to determine the branchpoint locations. A sequential 3D thinning algorithm reported by Palágyi et al. (2001) may serve as the basis for such skeletonization. To obtain the skeleton, a thinning function may be used to delete border voxels that can be removed without changing the topology of the tree. Such a thinning step may be applied repeatedly until no more points can be deleted. The thinning may be performed symmetrically and the resulting skeleton will lie in the middle of the cylindrically shaped airway segments.

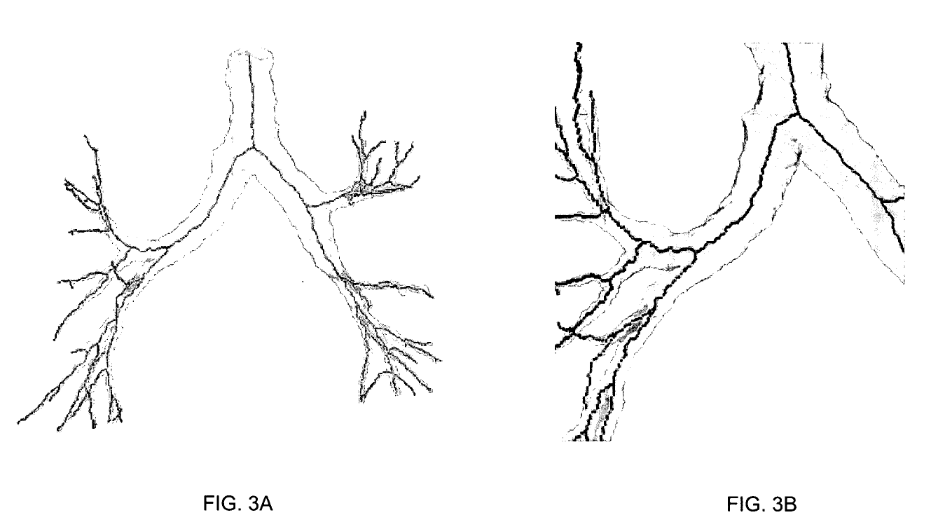

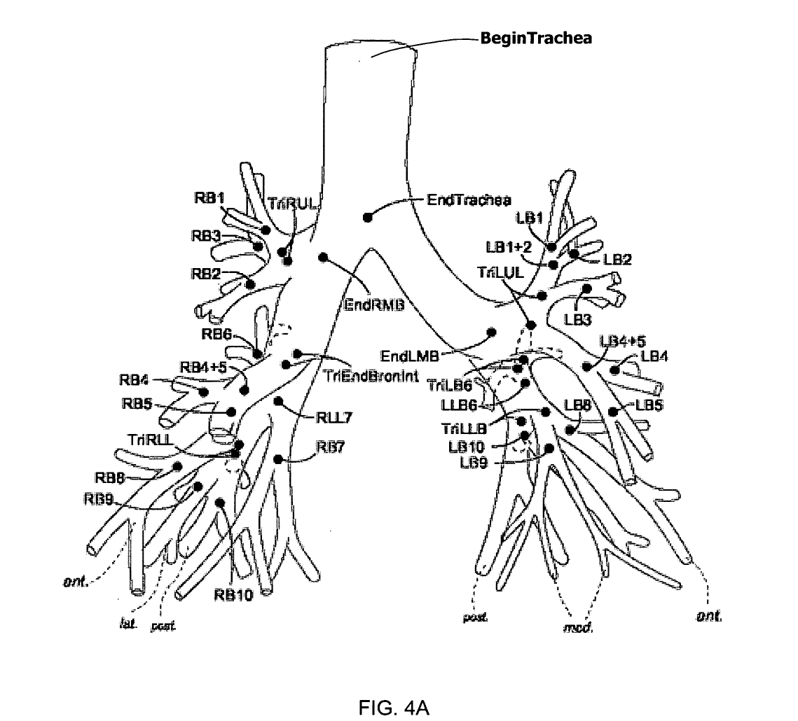

After completion of the thinning step, the skeleton may be smoothed (see Palágyi et al.), false branches may be pruned, the location of the branchpoints may be identified, and the complete tree may be converted into a graph structure using an adjacency list representation (see Palágyi et al.). The pruning and branchpoint location identification techniques described in U.S. patent application Ser. No. 11/122,924 filed May 5, 2005 and entitled “Methods and Devices for Labeling and/or Matching” may be used in this regard, which co-pending application is expressly incorporated by reference. FIGS. 3A and 3B show different views of a skeleton produced by the airway tree segmentation and skeletonization described in this section. Skeleton branchpoints may be identified as skeleton points with more than two neighboring skeleton points.

### Measurements of Airway Tree Geometry

In some embodiments, minor and major airway diameter, cross-sectional area, and wall-thickness are measured. The techniques described in section 5.4 “Methods” of Tschirren (which section is incorporated by reference) may be used in this regard. Measurements may be taken at every centerline voxel position. The inner and outer airway wall may be segmented simultaneously using 3D optimal surface detection based on the graph searching algorithm of Wu and Chen (2002). While inner wall surfaces are generally visible in CT images, outer airway wall surfaces are very difficult to segment due to their blurred and discontinuous appearance. Adjacent blood vessels further increase the difficulty of this task. By optimizing the inner and outer wall surfaces and considering the geometric constraints, a coupled-surfaces segmentation approach (Li et al., 2004) may produce good segmentation results for both airway wall surfaces in a robust manner. Unlike other optimal surface detection methods, Wu and Chen's algorithm does not suffer from exponentially growing computational complexity, which makes it run efficiently. A complete airway tree can be measured in about 3 minutes on a dual CPU 3 GHz machine. All measurement results may be stored in a hierarchical XML file that holds information about airway geometry, topology, and anatomical labels. This allows the user to easily access results for further processing.

### Anatomical Labeling of Airway Tree

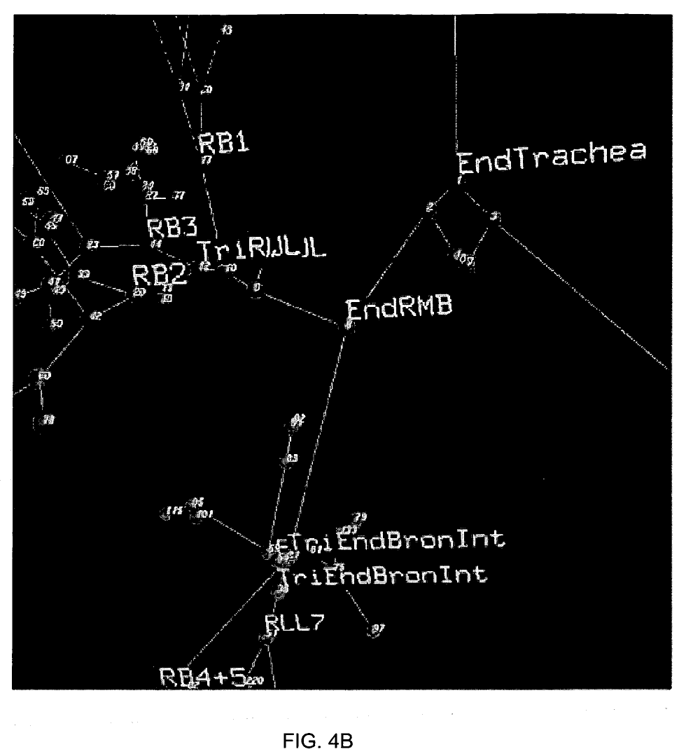

In some embodiments, the segmented airway tree may be anatomically labeled, such that 33 commonly used labels are assigned to the airway tree. FIG. 4A depicts a sample airway tree with branchpoints labeled anatomically. The tree is rooted at the branchpoint labeled BeginTrachea. The sub-tree rooted at TriRUL corresponds to the right upper lobe. Sub-trees rooted at RB4+5, TriEndBronInt, TriLUL and LLB6 correspond to the right middle lobe, right lower lobe, left upper lobe and left lower lobe, respectively.

The labels may be assigned by matching the graph representation of the tree against a population average. This population average may be created by averaging a set of trees that are hand-labeled by a human expert. The population average may incorporate the natural variability of the airway geometry and topology as it can be observed across the population. This will make the automated labeling robust against such variations. This labeling technique is described in U.S. patent application Ser. No. 11/122,924 filed May 5, 2005 and entitled “Methods and Devices for Labeling and/or Matching,” and that description is expressly incorporated by reference.

Not all false branches can automatically be eliminated during the skeletonization. However, the anatomical labeling module (or algorithm) may be aware of that and be able to tolerate false branches in almost all cases. FIG. 4B shows a detail view of a tree that has been automatically labeled. The anatomical labeling may be performed in hierarchical steps to reduce the runtime of the algorithm to a few seconds.

### Lobe Segmentation

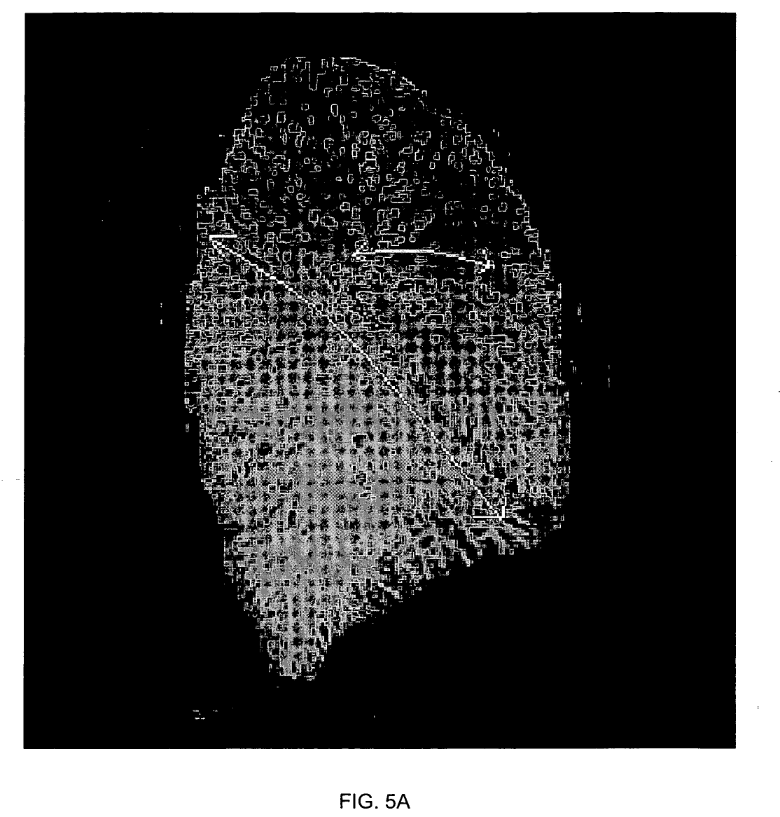

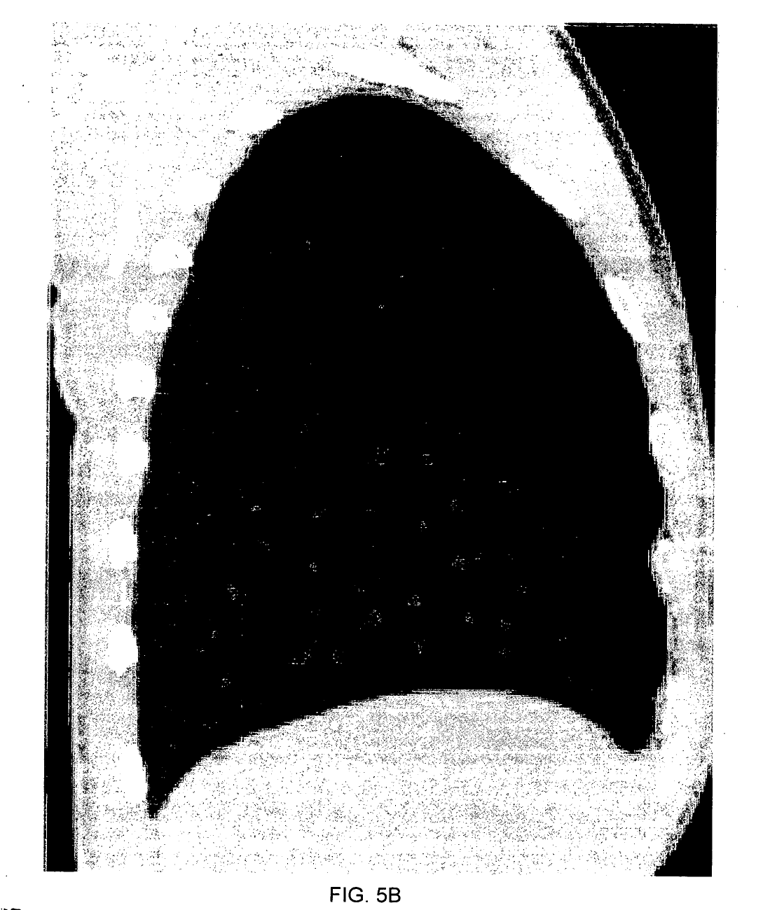

The human lungs are divided into five distinct anatomic compartments called lobes. The physical boundaries between the lobes are called the lobar fissures. The lobes can also be distinguished by the separate vascular and airway sub-trees supplying each lobe. FIG. 5A is a 3D rendering of a segmented vascular tree that shows the position of the fissures as a separation in the otherwise dense tree, providing an indirect method in some embodiments to estimate the lobar fissures.

In other embodiments, another way to segment the lobes includes segmenting the vascular tree, and computing a watershed transform on a distance map of the segmented vascular tree. The distance map may be determined (e.g., created) by assigning to every background voxel its distance to the closest foreground voxel, where “foreground” voxels belong to a vessel and “background” voxels are any other voxels within the lung. With the vascular tree segments corresponding to the topographical basins of the gray level image, the lobes may be segmented by placing seed points on the corresponding vascular sub-trees. Because the airway tree segments run close to the vascular tree segments for a number of generations, the seed point placement may be automated by using the anatomical description of the airway tree. Vascular tree points may be identified in a window (e.g., a window appearing on a display device) near the airway sub-tree branchpoints and endpoints, and those points may be used as seeds.

Using the approach to lobe segmentation described in the previous paragraph, a close approximation to the lobar fissures should be achieved (see FIG. 5B), especially the oblique fissures in both lungs. This result may then be used to narrow the search region for more sophisticated gray level search techniques to locate the actual fissures.

The lobe segmentation technique below is one manner of carrying out lobe segmentation consistent with some embodiments of the present methods, devices and systems.

3.1 Lobe Segmentation Introduction

Lobe segmentation is the automatic identification of the five lung lobes: left upper, left lower, right upper, right middle and right lower lobes from chest CT scans. The right upper and right middle lobes are separated by the horizontal fissure. The right upper and right middle lobes together are separated from the right lower lobe by the right oblique fissure. The left upper lobe is separated from the left lower lobe by the left oblique fissure. See FIGS. 1H and 1I.

3.1.1 Inputs

16-bit CT image object, 8-bit lung smoothing image object, 8-bit vascular tree segmentation image object, 8-bit airway tree segmentation image object, Airway tree XML file.

3.1.2 Outputs

Analyze 8-bit volume with separate labels for each lobe.

3.1.3 Orientation

The lungs are assumed to be oriented such that, in the memory representation, X increases from the right of the patient to the left, Y increases from the front of the patient to the back, and Z increases from the head to the feet.

3.2 Algorithm

A lobe segmentation flow chart appears in FIG. 1J. The lobe segmentation algorithm comprises the following main steps:
1. Computing a distance map on the vessel segmentation result and combine with raw data.
2. Computing a watershed transform on the distance map image.
3. Extracting markers/seed points based on the airway tree anatomical labeling and other information.
4. Using extracted markers for watershed reconstruction, to segment the lobes.
   3.2.1 Vessel Distance Map

The lobe segmentation algorithm is based on computing a watershed transform on a distance map of the vessel segmentation. The guiding principle is that the fissures are characterised by an absence of vessels, and hence can be thought of as local maxima in the distance map. If we consider the vessels and airways as the basins of the image topography and spread labels from these basins, we can expect that the labels corresponding to the different lobes to meet at or near the fissures. So our distance map image should correspond to basins at the vessels and airways, and peaks corresponding to the fissures. It is calculated as follows:
Scale up the raw image I, so that the minimum value within the lung mask is 0.
Within the lung mask compute vessel CT value maxima, airway CT value minima.
Compute a chamfer distance map on the vessel mask, using the standard 3×3×3 kernel (Borgefors, 1986). The distance is computed from every background voxel to nearest vessel voxel.
Combine the distance map and original image according to the following scheme:

$${O}_{v} = \{ \begin{matrix}{V}_{max} - {I}_{v} & if v ∈ V ; \\ {I}_{v} - {A}_{min} & if v ∈ A ; \\ {V}_{max} + {I}_{v} \text{\vert} + α * {D}_{v} & otherwise .\end{matrix}$$
O refers to the output image and D is the distance map. V and A are the sets of segmented vessel and airway voxels respectively. Vₘₐₓ is the maximum density value of any segmented vessel voxel, and Aₘᵢₙ is the minimum density value of any segmented airway voxel. α is the index used for combining the original image and distance map image. We use a value of 10, for α.

The above method ensures that the airways and vessels correspond to the basins and the fissures are the peaks or watersheds(also called merging candidates), of the 3-D topography of the image.

3.2.2 Watershed Transform

The watershed transform algorithm used is the Interactive Watershed Transform (IWT) algorithm proposed by (Hahn and Peitgen, 2003), for its efficiency and real-time interactivity. It computes the basins and merges from the input image and arranges them hierarchically in a tree. FIG. 1K shows the basins, bᵢ and merges, mᵢ for a 1-D gray level topography, which are ordered according to their depth. The algorithm is described below:
Require: Input image I[pεD], N(p) specifying connected neighbors of voxel p, e.g. 4,6,8 connectivity.
Ensure: Output Table of atomic basins B=∪bᵢ where:
   g[i] the lowest gray level value of basin bᵢ
   r[i] is the reference by index to the deepest connected basin
   l[i] is the basin label
Ensure: Output Table of merging candidates M=∪mj, where:
   k[j] index of basin candidate bₖ that is to be merged
   a[j] index of the deeper basin bₐ that is to be merged to bₖ
   g[j] gray level of merging event mⱼ

Initialize:

```
Work Image W(p) [symbol] 0,∀ₚ ∈ D
β [symbol] 0 //basin counter
μ [symbol] 0 //merging counter
Sort voxels by gray level in ascending order and store in container S
for v = 1 to size of S do
  p [symbol] S[v]
  Bₚ [symbol] {bₖ ∈ B| ∃_{q} ∈ N(p) [symbol] W[q] = k} //neighboring basins
  if Bp = 0 ; then
    β [symbol] β + 1
    g[β] [symbol] gray level value of p
    r[β] [symbol] β
    W[p] [symbol] β
  else
    α [symbol] arg min{BASE(k) | bₖ ∈ Bₚ}|
    W[p] [symbol] α //basin counter
    for ∀bₖ ∈ Bₚ do
      if BASE(k) ≠ BASE(α) then
        r[BASE(k) [symbol] r[BASE(α)]
        μ [symbol] μ + 1
        k[μ] [symbol] k
        a[μ] [symbol] α
        g[μ] [symbol] gray level value of p
      end if
    end for
  end if
end for
function BASE(k)
if r[k] ≠ k then
    k [symbol] BASE(r[k])
end if
return k
```

Once the watershed transform has been computed, the basins can be merged according to selected markers in real-time, using the following algorithm:

Require: h_{pf}, the pre-flooding height to control basin merging, and the set of markers MARK.

```
for ∀bₖ ∈ B do
  l[k] [symbol] 0
  r[k] [symbol] k
end for
// Label basins with marker labels
for ∀m ∈ MARK do
  b [symbol] W[m]
  l[b] [symbol] label of m
end for
// Consider each merging candidate from bottom to top
for μ = 1 to size of M do
  MERGE(μ, h_{pf})
end for
// A single merge of 2 basins
k [symbol] BASE(k[μ])
a [symbol] BASE(a[μ])
function MERGE(μ, h_{pf})
if h_{pf}≧ g[μ]−g[k] then
  if l[a] == 0 α l[k] == 0α == l[k] then
    r[k] = = a
  end if
  if l[a] = = 0 then
    l[a] = l[k]
  end if
end if
// Label lookup for voxel p
function LABEL(p)
k [symbol] W[p]
if l[k] == 0 [symbol] r[k] ≠ k then
  l[k] [symbol] l[BASE(k)]
end if
return l[k]
```

NOTE: The basin and merging tables, referred to in the algorithm above, have three different fields for each basin and merging index. The tables were implemented as vector of vectors (C++ Standard Template Library), thus dynamic allocating memory for each new basin and merging candidate.
3.2.3 Marker Selection

The IWT requires markers for basin merging. The markers for basin merging are selected automatically, from the airway tree anatomical labeling. The airway tree XML file is read and the tree structure is extracted. The airway branchpoints corresponding to the roots of the different lobar sub-trees are TriLUL, LLB6, TriRUL, RB₄+5 and TriEndBronInt, which correspond to the left upper lobe, left lower lobe, right upper lobe, right middle lobe and the right lower lobe respectively (FIG. 1L). Using the formal graph description extract the whole sub-tree for a given lobe, by doing a breadth-first search from the corresponding lobar root. Once the sub-trees are extracted, set all centerline points as markers for that give lobe. Further, construct a window around each branchpoint of the sub-tree and pick all points in the windows as markers. This is done to pick the lower generation vascular segments that are located near the airway tree segments. The window sizes for picking the markers are different for each lobe, and are selected according to a-priori shape information. The following are the windowing limits for each lobe, centered around the corresponding branchpoints, in pixels:

| | Xₘᵢₙ | Xₘₐₓ | Yₘᵢₙ | Yₘₐₓ | Zₘᵢₙ | Zₘₐₓ |
|---|---|---|---|---|---|---|
| Left upper | −20 | 10 | −30 | 5 | −3 | 0 |
| Left lower | −10 | 20 | −5 | 30 | 0 | 3 |
| Right upper | −20 | 20 | −30 | 15 | −3 | 0 |
| Right middle | −15 | 20 | −20 | 10 | −3 | 0 |
| Right lower | −10 | 20 | −5 | 30 | 0 | 3 |

Additionally select some extra markers from a-priori shape information for the lobes. Take transverse slice number 30 from the top of the lung and pick all vascular segments as markers for the upper lobes for each lung.
3.2.4 Watershed Reconstruction/Basin Merging

After selecting all markers, the lobes can be segmented using the basin merging procedure described above. It should be noted that if the segmentation is not satisfactory, manually selected markers can be placed and the basin merging redone interactively and in real-time.

The oblique fissure segmentation technique and the horizontal fissure segmentation technique described below represent suitable, example ways of carrying out oblique and horizontal fissure segmentation (more broadly, fissure segmentation) consistent with some embodiments of the present methods, devices and systems:

4.1 Oblique Fissure Segmentation Introduction

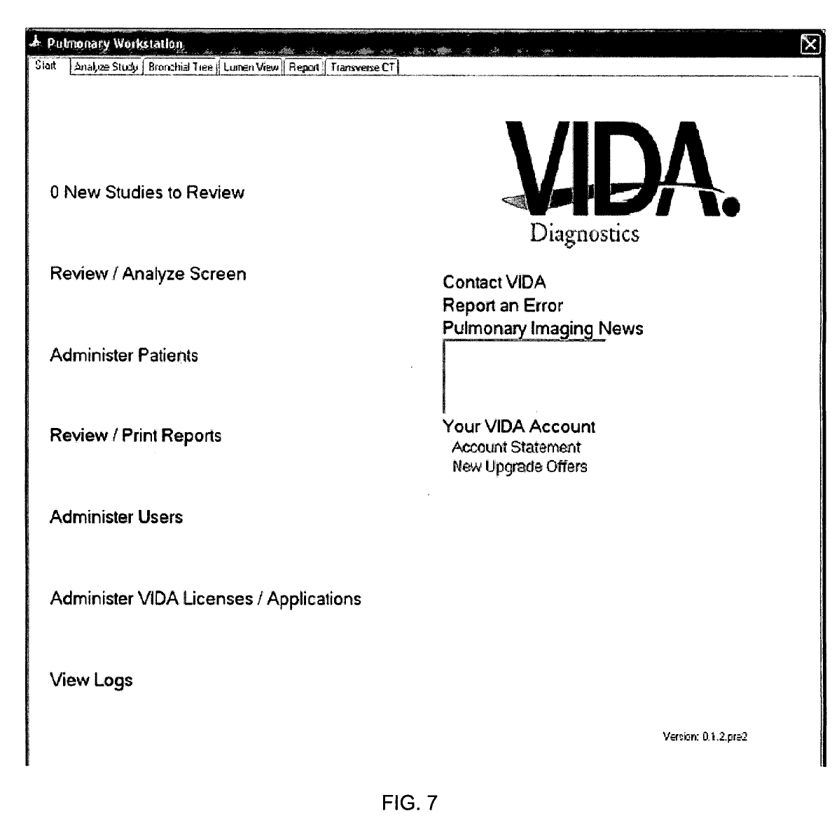

Oblique fissure segmentation is the automatic identification of the right and left oblique fissures. The fissure ROI is determined from the approximate lobar boundaries (FIG. 7.1) found by the watershed segmentation algorithm described in sections 3.1-3.2.4 above, and we find the exact fissure surface using 3-D graph search.

4.1.1 Inputs

16-bit CT image object, 8-bit smoothed lung image object, 8-bit segmented lobe image object (chapter 6), 8-bit output image object.

4.1.2 Outputs

Analyze 8-bit volume with separate labels for upper and lower lobes (for the right lung, the upper and middle lobes are combined under a single upper lobe label).

4.1.3 Orientation

The lungs are assumed to be oriented such that, in the memory representation, X increases from the right of the patient to the left, Y increases from the front of the patient to the back, and Z increases from the head to the feet.

4.2 Algorithm

The oblique fissure segmentation algorithm comprises the following main steps:
1. Compute the fissure ROI from watershed segmentation result.
2. Rotate ROI to make the fissure vertical.
3. Compute ridgeness measure for all XY slices of ROI, to enhance fissures.
4. Define cost function as the sum of ridgeness value and raw data value.
5. Detect fissure using 3-D optimal surface search, as Y=f(X, Z).
6. Labeling upper and lower lobes.
   4.2.1 Finding fissure ROI

The fissure ROI is found based on the lobe segmentation result obtained by the method described in sections 3.1-3.2.4 above. First, we extract all upper lobe (and middle lobe, for right lung) voxels with at least one 6-connected lower lobe voxel. This constitutes our approximate fissure point set. From this point set we compute an approximate euclidean distance map as described in (Borgefors, 1986), which gives us the distance of each background voxel to the closest foreground voxel. We then compute a fissure mask with three labels: ROI1, ROI2 and LUNG. ROI1 refers to all lung voxels which are at a distance≦d1, and LUNG refers to all other lung voxels. ROI2 refers to all non-lung voxels which are at a distance≦d2. We use the values d1=6.23538 mm and d2=4.1569 mm. The approximate euclidean distance map values are converted to metric values by assuming that \sqrt{xᵢ²+yᵢ²+zᵢ²} mm˜5 Chamfer distance units. xᵢ, yᵢ and zᵢ are the image voxel dimensions. FIG. 1O shows a sample transverse slice from the left lung showing the three labels.

4.2.2 Rotate ROI Image

The oblique fissure is tilted by a certain angle away from the Z(vertical) axis, as can be seen in FIGS. 1M and IN. For speeding up the 3-D graph search later on, we want to rotate the ROI to make it vertical and hence reduce the Y dimension of the fissure bounding-box. To compute this angle of rotation we perform the following steps:
Extract the sagittal slice at X=(Xₘᵢₙ+2*Xₘₐₓ)/3 for the left lung and at X=(2*Xₘᵢₙ+Xₘₐₓ)/3 for the right lung, where Xₘᵢₙ and Xₘₐₓ are the minimum and maximum X co-ordinates of the lung bounding box.
Fit a line, in the least squares sense, to the fissure points on that slice, as shown in FIG. 1P.
The angle that this line makes with the Z axis, a (FIG. 1P), is the angle by which we rotate the ROI image.
The axis of rotation is a line parallel to the X axis, and passing through the centre of the YZ plane of the lung bounding box.

To rotate the raw image, we use tri-linear interpolation, along with re-sampling to make the voxels isotropic. Let

$$R = [ \begin{matrix}cos ( a ) & sin ( a ) \\ - sin ( a ) & cos ( a )\end{matrix} ]$$

be the forward rotation matrix, and

$$R = [ \begin{matrix}cos ( a ) & - sin ( a ) \\ sin ( a ) & cos ( a )\end{matrix} ]$$

be the inverse rotation matrix. If xᵢ, yᵢ and zᵢ are the original voxel dimensions, set new voxel dimensions for rotated image as xₒ=yₒ=zₒ=min(xᵢ, yᵢ, zᵢ). The rotation scheme can then be described as follows:

Require: Let I be original image, F input mask image, and O be output raw image and M be the output mask image

```
// Set axis of rotation parallel to X, passing through center of the Y Z
plane
y_{c} [symbol] (y dim×yᵢ)/2
z_{c} [symbol] (z dim×zᵢ)/2
y_{o ff} [symbol] (Rᵢₙᵥ[0][0]×y_{c}+Rᵢₙᵥ[0][1]×z_{c}−y_{c})
z_{o ff} [symbol] (Rᵢₙᵥ[1][0]×y_{c}+Rᵢₙᵥ[1][1]×z_{c}−z_{c})
for ∀v=(x,y,z) ∈ O do
  yₘ [symbol] y*yₒ
  zₘ [symbol] z*zₒ
  X [symbol] x*xₒ
  Y [symbol] (Rᵢₙᵥ[0][0]×yₘ+Rᵢₙᵥ[0][1]×zₘ−y_{off})
  Z [symbol] (Rᵢₙᵥ[1][0]×yₘ+Rᵢₙᵥ[1][1]×zₘ−z_{off})
  x_{f} [symbol] floor(X/xᵢ)
  y_{f} [symbol] floor(Y/yᵢ)
  z_{f} [symbol] floor(Z/zᵢ)
  a [symbol] X−x_{f}×xᵢ
  b [symbol] Y−y_{f}×yᵢ
  c [symbol] Z−z_{f}×zᵢ
  O(x,y,z) [symbol] (1−c)*(1−b)*(1−a)*I(x_{f},y_{f},z_{f})+(1−c)*(1−b)*(a)*I(x_{f}+1,y_{f},z_{f})+
  (1−c)*(b)*(1−a)*I(x_{f},y_{f}+1,z_{f})+(1−c)*(b)*(a)*I(x_{f}+1,y_{f}+1,z_{f})+(c)*(1−b)
  *(1−a)*I(x_{f},y_{f},z_{f}+1)+(c)*(1−b)*(a)*I(x_{f}+1,y_{f},z_{f}+1)+(c)*(b)*(1−a)
  *I(x_{f},y_{f}+1,z_{f}+1)+(c)*(b)*(a)*I(x_{f}+1,y_{f}+1,z_{f}+1)
  xx [symbol] (int)(X/xᵢ=0.5)
  yy [symbol] (int)(Y/yᵢ=0.5)
  zz [symbol] (int)(X/zᵢ=0.5)
  M(x,y,z) [symbol] F(xx,yy,zz)
end for
```

After rotation, we find the bounding-box for the label ROI1, as shown in FIG. 1Q. This bounding box defines the graph within which we search for the optimal surface.
4.2.3 Ridgeness Map

We enhance the fissures by computing the ridgeness value. The ridge detector is based on the MLSEC-ST (Multilocal level set extrinsic curvature and its enhancement by structure tensors) (Li et al. II, 2004). The ridgeness map is calculated for all XY slices of the fissure bounding box.

4.2.4 Cost Function

The cost function is defined in the following manner:

$${C}_{v} \{ \begin{matrix}{I}_{v} + {R}_{v} & if v ∈ ROI 1 ; \\ - 100 & if v ∈ ROI 2 ; \\ - 2000 & if v ∈ LUNG ; \\ - 1000 & otherwise .\end{matrix}$$

I refers to the rotated input CT image. R is the ridgeness map of I. FIG. 1R shows a sample sagittal slice from the cost function. It should be noted that setting very low values to the region outside the ROI contributes towards a reduced run-time for the optimal graph search, but ROI2 is given a higher cost function value to cause a smoother transition of the optimal surface from the inside of the lung to the outside.
4.2.5 3-D Graph Search

After defining the cost function, we find the optimal 3-D surface f(X, Z), within the graph defined by the image −C. The algorithm proposed by (Li et al. II, 2004) is used, which finds the optimal surface by transforming the problem to computing the minimum s-t cut in a derived graph. Detailed description of the algorithm is presented in (Li et al. II, 2004). The different parameters for the optimal surface search, that are used for this application are:
The graph representation used is the implicit arc representation, and the Boykov-Kolmogorov maximum-flow based algorithm is selected.
The smoothness parameter is set to 1 for both X and Z dimensions, which corresponds to maximum smoothness.
The circularity parameter is set to 0 for both X and Z.
   4.2.6 Labeling Output Volume

The optimal surface search returns a single voxel wide connected surface with label 255, and 0 elsewhere. For a detected fissure point (x_{f}, y_{f}, z_{f}), all lung voxels (x_{f}, y, z_{f})|y<=y_{f} are assigned to the upper lobe. Similarly, all lung voxels (x_{f}, y, z_{f})|y>y_{f} belong to the lower lobe. Using this information, the following steps are performed to label the lobes:
Input: Image I, Segmented fissure image
Output: Image 0, with lobes labeled
initialize empty queue of voxels
for each fissure voxel x,y,z
   push x,y,z and x,y+1,z into the queue
   0(x,y,z)=upperlobe
   0(x,y+1,z)=lowerlobe
do
   pop voxel from queue
   label=0(voxel)
   for each 6 connected lung neighbor of voxel
      if it is unlabeled
         0(neighbor)=label
         push neighbor onto queue
while queue is not empty

Next, the labels are rotated back to the initial orientation, using nearest neighbor interpolation. Any remaining unlabelled lung voxels are labeled using the same algorithm as above.

5.1 Horizontal Fissure Segmentation Introduction

Horizontal fissure segmentation is the automatic identification of the right and left oblique fissures. The fissure ROI is determined from the approximate lobar boundaries (FIG. 1S) found by the watershed segmentation algorithm described in sections 3.1-3.2.4 above, and we find the exact fissure surface using 3-D graph search. The middle lobe is a subset of the right upper lobe found in sections 4.1-4.2.6 above.

5.1.1 Inputs

16-bit CT image object, 8-bit segmented oblique fissure image object (sections 4.1-4.2.6), 8-bit segmented lobe image object (sections 3.1-3.2.4), 8-bit output image object.

5.1.2 Outputs

Analyze 8-bit volume with separate labels for upper, middle and lower lobes.

5.1.3 Orientation

The lungs are assumed to be oriented such that, in the memory representation, X increases from the right of the patient to the left, Y increases from the front of the patient to the back, and Z increases from the head to the feet.

5.2 Algorithm

The oblique fissure segmentation algorithm comprises the following main steps:
1. Compute the fissure ROI from watershed segmentation result.
2. If the input volume is not isotropic, resample using tri-linear interpolation to compute isotropic volume.
3. Compute ridgeness measure for all YZ slices of ROI, to enhance fissure.
4. Define cost function as the sum of ridgeness value and raw data value.
5. Detect fissure using 3-D optimal surface search, as Z=f(X, Y).
6. Labeling upper and middle lobes.
   5.2.1 Finding Fissure ROI

The fissure ROI is found based on the lobe segmentation result obtained by the method described in sections 3.1-3.2.4 above. First, we extract all right upper lobe voxels with at least one 6-connected middle lobe voxel. This constitutes our approximate fissure point set. From this point set we compute an approximate euclidean distance map as described in (Borgefors, 1986), which gives us the distance of each background voxel to the closest foreground voxel. We then compute a fissure mask with three labels: ROI1, ROI2 and LUNG. ROI1 refers to all lung voxels which are at a distance≦d1, and LUNG refers to all other lung voxels. ROI2 refers to all non-lung voxels which are at a distance≦d2. We use the values d1=6.23538 mm and d2=4.1569 mm. The approximate euclidean distance map values are converted to metric values by assuming that \sqrt{xᵢ^{2′}+yᵢ²+zᵢ²} mm˜5 Chamfer distance units. xᵢ, yᵢ and zᵢ are the image voxel dimensions. FIG. 1T shows a sample transverse slice from the left lung showing the three labels.

5.2.2 Re-sample ROI Image

If the input CT volume is non-isotropic, we resample the dataset to make it isotropic. If xᵢ, yᵢ and zᵢ are the original voxel dimensions, set new voxel dimensions for rotated image as xₒ=yₒ=zₒ=min(xᵢ, yᵢ, zᵢ). The interpolation scheme can then be described as follows:

Require: Let I be original image, F input mask image, and O be output raw image and M be the output mask image

```
for ∀v=(x,y,z) ∈ O do
  X [symbol] x*xₒ
  Y [symbol] y*yₒ
  Z [symbol] z*zₒ
  x_{f} [symbol] floor(X/xᵢ)
  y_{f} [symbol] floor(Y/yᵢ)
  z_{f} [symbol] floor(Z/zᵢ)
  a [symbol] X−x_{f}×xᵢ
  b [symbol] Y−y_{f}×yᵢ
  c [symbol] Z−z_{f}×zᵢ
  O(x,y,z) [symbol] (1−c)*(1−b)*(1−a)*I(x_{f},y_{f},z_{f})+(1−c)*(1−b)*(a)*I(x_{f}+1,y_{f},z_{f})+
  (1−c)*(b)*(1−a)*I(x_{f},y_{f}+1,z_{f})+(1−c)*(b)*(a)*I(x_{f}+1,y_{f}+1,z_{f})+(c)*(1−b)
  *(1−a)*I(x_{f},y_{f},z_{f}+1)+(c)*(1−b)*(a)*I(x_{f}+1,y_{f},z_{f}+1)+(c)*(b)*(1−a)
  *I(x_{f},y_{f}+1,z_{f}+1)+(c)*(b)*(a)*I(x_{f}+1,y_{f}+1,z_{f}+1)
  xx [symbol] (int)(X/xᵢ=0.5)
  yy [symbol] (int)(Y/yᵢ=0.5)
  zz [symbol] (int)(Z/zᵢ=0.5)
  M(x,y,z) [symbol] F(xx,yy,zz)
end for
```

After re-sampling, we find the bounding-box for the label ROI1, as shown in FIG. 1U. This bounding box defines the graph within which we search for the optimal surface.

5.2.3 Ridgeness Map

We enhance the fissures by computing the ridgeness value. The ridge detector is based on the MLSEC-ST (Multilocal level set extrinsic curvature and its enhancement by structure tensors) (Lopez et al., 2000). The ridgeness map is calculated for all YZ slices of the fissure bounding box.

5.2.4 Cost Function

The cost function is defined in the following manner:

$${C}_{v} \{ \begin{matrix}{I}_{v} + {R}_{v} & if v ∈ ROI 1 ; \\ - 100 & if v ∈ ROI 2 ; \\ - 2000 & if v ∈ LUNG ; \\ - 1000 & otherwise .\end{matrix}$$

I refers to the rotated input CT image. R is the ridgeness map of I. FIG. 1V shows a sample sagittal slice from the cost function. It should be noted that setting very low values to the region outside the ROI contributes towards a reduced run-time for the optimal graph search, but ROI2 is given a higher cost function value to cause a smoother transition of the optimal surface from the inside of the lung to the outside.
5.2.5 3-D Graph Search

After defining the cost function, we find the optimal 3-D surface f(X, Y), within the graph defined by the image −C. The algorithm proposed by (Li et al. II, 2004) is used, which finds the optimal surface by transforming the problem to computing the minimum s-t cut in a derived graph. Detailed description of the algorithm is presented in (Li et al. II, 2004). The different parameters for the optimal surface search, that are used for this application are:
The graph representation used is the implicit arc representation, and the Boykov-Kolmogorov maximum-flow based algorithm is selected.
The smoothness parameter is set to 1 for both X and Y dimensions, which corresponds to maximum smoothness.
The circularity parameter is set to 0 for both X and Y.
   5.2.6 Labeling Output Volume

The optimal surface search returns a single voxel wide connected surface with label 255, and 0 elsewhere. For a detected fissure point (x_{f}, y_{f}, z_{f}), all lung voxels (x_{f}, y_{f}, z)|z<z_{f} are assigned to the upper lobe. For a detected fissure point (x_{f}, y_{f}, z_{f}), all lung voxels (x_{f}, y_{f}, z)|z<z_{f} are assigned to the upper lobe. Similarly, all lung voxels (x_{f}, y_{f}, z)|z>=z_{f} belong to the middle lobe. Using this information, the following steps are performed to label the lobes:
Input: Image I, Segmented fissure image
Output: Image 0, with lobes labeled
initialize empty queue of voxels
for each fissure voxel x,y,z
push x,y,z−1 and x,y,z into the queue

$$0 ( x , y , z - 1 ) = upperlobe$$

$$0 ( x , y , z ) = middlelobe$$
do
   pop voxel from queue
   label=0(voxel)
   for each 6 connected lung neighbor of voxel
      if it is unlabeled
         0(neighbor)=label
         push neighbor onto queue
while queue is not empty

Next, the labels are transferred to the original volume, using nearest neighbor interpolation. Any remaining unlabelled lung voxels are labeled using the same algorithm as above.

### Low Attenuation Cluster Analysis

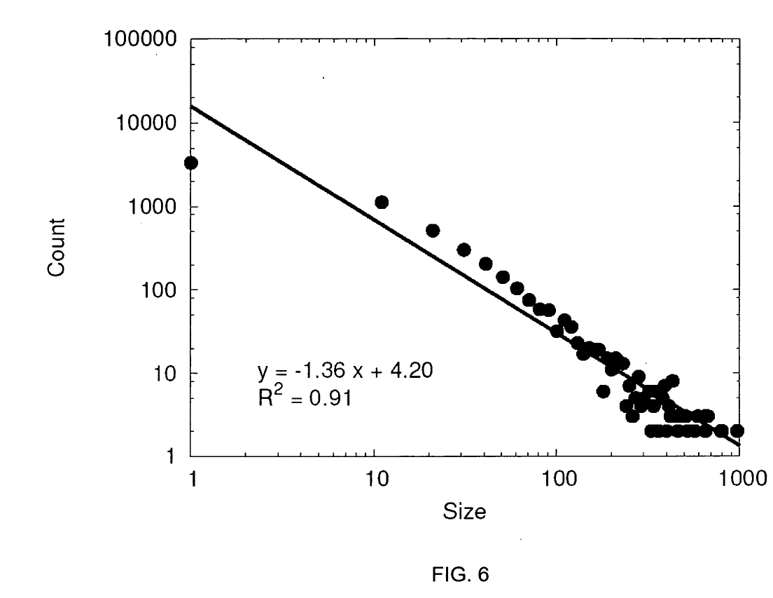

In some embodiments (e.g., those in which emphysematic tissue is the target tissue), low attenuation clusters (LACs) representative of disease may be found by thresholding the gray level images in a dataset of images making up an anatomical region of interest (e.g., the lungs). A low attenuation cluster is one type of potential treatment region and may, if selected as described below in conjunction with Pathfinder, be characterized as a target treatment region. The dataset of images may be obtained using any of the imaging modalities discussed above. When some form of CT is used, the left and right lung may be distinguished by masking the CT volume with the lung segmentation result. The threshold result may be traversed and one-voxel bridges that connect neighboring clusters may be removed. The volume of each cluster may then be measured and the histogram of the cluster sizes may be computed. Taking the logarithm of both the ordinate (count) and abscissa (cluster size) leads to a linearization of the histogram (see FIG. 6). The histogram is then represented by the linear function log(c)=α·log(s)+β with s and c being the cluster size and the count, respectively. The values for α and β are determined by a simple linear interpolation. The α value is a reliable indicator for the degree of emphysema because, compared with normal subjects, emphysema patients have a proportionally higher count of big air-clusters, which affects the slope α (Mishima et al., 1999). Thus, the slope a is a good indicator for the degree of emphysema.

### Identifying Relevant Airway Segments

Some embodiments include finding a feeding airway to a region t of the lung. This may be done for different reasons, including: 1) to determine which airway path is supplying air to region t; and 2) to determine which airway path is leading closest to region t such that the region can be accessed by a medical device capable of traversing the airway tree (e.g., an endoscope or a bronchoscope). These two reasons are examples of conditions that qualify as therapeutically linking a potential location for a therapeutic device (e.g., a one-way valve or a stent) to a target treatment region (e.g., a LAC representing emphysematic lung tissue).

The target t may be any point of interest inside a given lung, e.g., a low attenuation cluster (in the case of emphysema), a suspected or confirmed lung nodule, a lymph node, or any other location within the lung that can be specified by a coordinate. The target t may be specified by a single point (e.g., the center of a lung nodule), or it may be specified by a set of coordinates (e.g., the coordinates of all the voxels that are located inside a nodule, or the coordinates of all the voxels that are located on the surface of a nodule). The set T may be defined to contain all the coordinates of the points that are contained within region t.

An example set of steps that may be performed to identify the feeding airway segments to a target region within a lung comprise:

a) Identifying the lung lobe(s) in which the coordinates in T are located (described below as “lobe(s) of interest”). The lobe(s) may be defined by the result of the lobar segmentation described above. The lobar segmentation algorithm may assign every voxel within the volumetric image scan (e.g., the dataset of images) to an anatomical lobe (or several anatomical lobes if no definitive assignment can be made due, for example, to an incomplete fissure line).

b) Finding all airway-tree segments (ats) that have the majority of their volume located within the lobe or lobes identified in step a). This step may be conducted by traversing the segmented airway branch-by-branch and determining if a given branch (up to all branches) is located in one of the lobes of interest.

c) For every airway-tree segment ats identified in step b), computing the distance to every point in T. Each such distance may be computed from only one single point within a given ats, or, alternatively, distances may be computed from several or all points within a given ats. When the “only one point” technique is used, the endpoint of every terminal branch may be used to compute the distance to the target t in order to find the terminal branch with the shortest associated distance. Computing the distance from multiple points within a given ats may be used to find the closest point on the surface of any ats to a given target t in cases of, for example, planning for a needle biopsy of that target. Every distance that is computed (e.g., found or determined) may be stored together with its associated ats and its associated point within that ats.

Such distances may be computed in any suitable way. For example, if the goal of the procedure is the planning of, for example, a needle-biopsy, then an Euclidean distance measure may be used. Alternatively, for better computational performance, an integer-based measure such as a city-block distance may be used instead of the Euclidean distance measure. The gray-value of the volumetric image scan of the lung may be factored into each distance measure such that gray-values that indicate a high air content represent shorter distances. In this regard, air-rich tissue may be favored over tissue with a low air content.

d) Sorting the distances determined in step c) and finding the N shortest ones. The value of N depends on the application and may be adjusted by the user.

e) For each point p within the airway tree that is associated with the N shortest distances found in step d), finding the shortest path between the points q and p, where point q may be the origin of the airway tree (the top of the trachea), or any other user-defined point.

### Example Implementations

Some (up to all) of the steps described in the sections above may be implemented in the form of software. The software may be stored on any suitable computer readable media, such as any suitable form of memory or data storage device, including but not limited to hard disks, removable magnetic disks, CDs, DVDs, optical disks, magnetic cassettes, flash memory cards, Bernoulli cartridges, USB drives, and the like. In such embodiments, the invention may be characterized as computer readable media having machine readable instructions for performing certain step(s).

Some (up to all) of the steps described in the sections above may be implemented using a computer having a processor (e.g., one or more integrated circuits) programmed with firmware and/or running software. Some (up to all) of the steps described in the sections above may be implemented using a distributed computing environment. In a distributed computing environment (e.g., a computer system), multiple computers may be used, such as those connected by any suitable number of connection mediums (e.g., a local area network (LAN), a wide area network (WAN), or other computer networks, including but not limited to Ethernets, enterprise-wide computer networks, intranets and the Internet).

Each step or group of steps identified by a bolded heading above may be coded as a standalone plug-in (e.g., a module) having a well-defined interface that may be integrated with other modules, and/or with a user interface (e.g., a graphical user interface). Standard-conforming C++ may be used. Well-established cross-platform libraries such as OpenGL and Boost may be utilized to execute embodiments of the present methods, devices and systems. Multi-threading may be used wherever applicable to reduce computing time on modem multiprocessor based hardware platforms. All modules may be written to enable them to be compiled on all common platforms (e.g., Microsoft®, Linux®, Apple Macintosh® OS X, Unix®).

One example software package, or application, that may be used to practice some embodiments of the steps described above will be referred to as “Pulmonary Workstation” in this disclosure. Those of ordinary skill in the art having the benefit of this disclosure will be able to write code (machine readable instructions) without undue experimentation for accomplishing the features of Pulmonary Workstation (including the graphical user interface) described below and shown in the figures. Pulmonary Workstation may be configured using the techniques disclosed in sections 1.1-5.2.6 above and the following sections pertaining to “Display,” “Regression Testing,” and “Coordinate System” disclosed below:

6 Display

6.1 Gamma Correction & 16 Bit to 8 Bit Conversion

Given an input voxel vᵢ (12 bit grayvalue) with a minimum grayvalue a and a maximum grayvalue b. Convert to a 8 bit grayvalue vₐ using gamma correction γ:

$${v}_{o} = 255 \cdot {1}^{- \frac{1}{γ}} \cdot {( \frac{{v}_{i} - a}{b - a} )}^{\frac{1}{γ}}$$

with explanations:
vₒ=255—endresult in range 0 . . . 255;

$${1}^{- \frac{1}{γ}}$$
—scale term to the right to 0 . . . 1;

$$\frac{{v}_{i} - a}{b - a}$$
—scale input to range 0 . . . 1;

$$\frac{1}{γ} - gamma correction .$$
—gamma correction.
γ=1 results in a linear conversion. γ>1 brightens the image. 0<γ<1 dims the image.
   7 Regression Testing
   7.1 Introduction, Definitions, Goals

Regression testing validates the output of an algorithm against a reference output. The current output of the algorithm is compared with the reference output, and a decision is made if the two outputs are identical. If the two outputs are identical within a pre-defined tolerance then the regression test returns as passed, otherwise it returns as failed. The passed/failed decision is made automatically, without any human intervention.

Every algorithm (in particular, every interface of every plugin) has an own regression test defined. Regression tests can be run in two different modes:

Manually executed local test. When working on a particular interface the developer can execute a regression test on that particular interface. The result of the regression test is printed in a human readable form to terminal window. This allows the developer to repeatedly and quickly check the performance of an alogorithm while doing changes on it.

Automatically executed global test. An automatically executed global test may for example be scheduled to run on a nightly basis. This test usually executes many different regression tests (e.g., on pre-configured interfaces). Each individual regression test outputs its results in a machine-readable format. The controlling instance gathers the individual results and combines them into one report, e.g., a webpage comparable to the “Dashboard” used by the ITK project.

7.2 Regression Test Executable

regression_executable [-h -x -o FILE] INPUT
-h, —human
   Output result in human-readable format. Default.
-x, —xml
   Output result in XML format (machine readable format).
-o FILE
   Redirect output to FILE.
INPUT
   File that defines test cases: pairs of input- and reference-output, allowed tolerances, etc. Number and kind of parameters depend on algorithm to be tested.
      7.3 Test and Reference Datasets

The test and reference datasets are organized in the following directory structure/naming scheme:

For each test case . . .
1. One or several reference input datasets (e.g., CT dataset)
2. Expected output data
   7.3.1 Directory Structure, Naming Scheme
subjectXXX/
   subjectXXX.hdr
   subjectXXX.img.gz
   airwaytree_segmentation_{—}0001/
      subjectXXX_rgrow.hdr
      subjectXXX_rgrow.img.gz
   lung_segmentation_{—}0001/
   subjectXXX_lung.hdr
   subjectXXX_lung.img.gz
   subjectXXX_lung_smoothed.hdr
   subjectXXX_lung_smoothed.img.gz
      7.3.2 Storage
central file server—reference
rsync-ed to test machine(s)
   8 Coordinate System
   8.1 Orientation of DICOM Images

This section 8.1 is copied from “Medical Image Format FAQ—Part 2” (available on the World Wide Web at faqs.org/faqs/medical-image-faq/part2/), Section 2.2.2. The bracketed “Projection radiographs” and “Cross-sectional images” and the italics (for emphasis) have been added.

Another question that is frequently asked in comp.protocols.dicom is how to determine which side of an image is which (e.g. left, right) and so on. The short answer is that for projection radiographs this is specified explicitly using the Patient Orientation attribute, and for cross-sectional images it needs to be derived from the Image Orientation (Patient) direction cosines. In the standard these are explained as follows:
[Projection radiographs] “C.7.6.1.1.1 Patient Orientation. The Patient Orientation (0020,0020) relative to the image plane shall be specified by two values that designate the anatomical direction of the positive row axis (left to right) and the positive column axis (top to bottom). The first entry is the direction of the rows, given by the direction of the last pixel in the first row from the first pixel in that row. The second entry is the direction of the columns, given by the direction of the last pixel in the first column from the first pixel in that column. Anatomical direction shall be designated by the capital letters: A (anterior), P (posterior), R (right), L (left), H (head), F (foot). Each value of the orientation attribute shall contain at least one of these characters. If refinements in the orientation descriptions are to be specified, then they shall be designated by one or two additional letters in each value. Within each value, the letters shall be ordered with the principal orientation designated in the first character.”
[Cross-sectional images] “C.7.6.2.1.1 Image Position And Image Orientation. The Image Position (0020,0032) specifies the x, y, and z coordinates of the upper left hand corner of the image; it is the center of the first voxel transmitted. Image Orientation (0020,0037) specifies the direction cosines of the first row and the first column with respect to the patient. These Attributes shall be provide as a pair. Row value for the x, y, and z axes respectively followed by the Column value for the x, y, and z axes respectively. The direction of the axes is defined fully by the patient's orientation. The x-axis is increasing to the left hand side of the patient. The y-axis is increasing to the posterior side of the patient. The z-axis is increasing toward the head of the patient. The patient based coordinate system is a right handed system, i.e. the vector cross product of a unit vector along the positive x-axis and a unit vector along the positive y-axis is equal to a unit vector along the positive z-axis.”

Some simple code to take one of the direction cosines (vectors) from the Image Orientation (Patient) attribute and generate strings equivalent to one of the values of Patient Orientation looks like this (noting that if the vector is not aligned exactly with one of the major axes, the resulting string will have multiple letters in as described under “refinements” in C.7.6.1.1.1):

```
char * DerivedImagePlane : : getOrientation (Vector3D vector) {
  char *orientation=new char[4];
  char *optr = orientation;
  *optr=’\0’;
  char orientationX = vector .getX( ) < 0 ∀ ’R’ : ’L’;
  char orientationY = vector .getY( ) < 0 ∀ ’A’ : ’P’;
  char orientationZ = vector .getZ( ) < 0 ∀ ’F’ : ’H’;
  double absX = fabs(vector .getX( ) );
  double absY = fabs(vector .getY( ) );
  double absZ = fabs(vector .getZ( ) );
  int i ;
  for (i=0; i<3; ++i) {
    if (absX>.0001 && absX>absY && absX>abs Z) {
      *optr++=orientationX;
      absX=0;
    }
    else if (absY>.0001 && absY>absX && absY>abs Z) {
      *optr++=orientationY;
      absY=0;
    }
    else if (absZ>.0001 && absZ>absX && absZ>absY) {
      *optr++=orientationZ;
      absZ=0;
    }
    else break;
    *optr=’\0’;
  }
  return orientation;
}
```

### Pulmonary Workstation

Pulmonary Workstation may be configured (e.g., code may be written) such that starting the application brings the user to a startup screen as illustrated in FIG. 7. Several links may be provided, such as those listed on the left hand side of FIG. 7, but for this disclosure, only the “Review/Analyze Screen” and “Administer Patients” links will be described.

Choosing a Patient from the Database

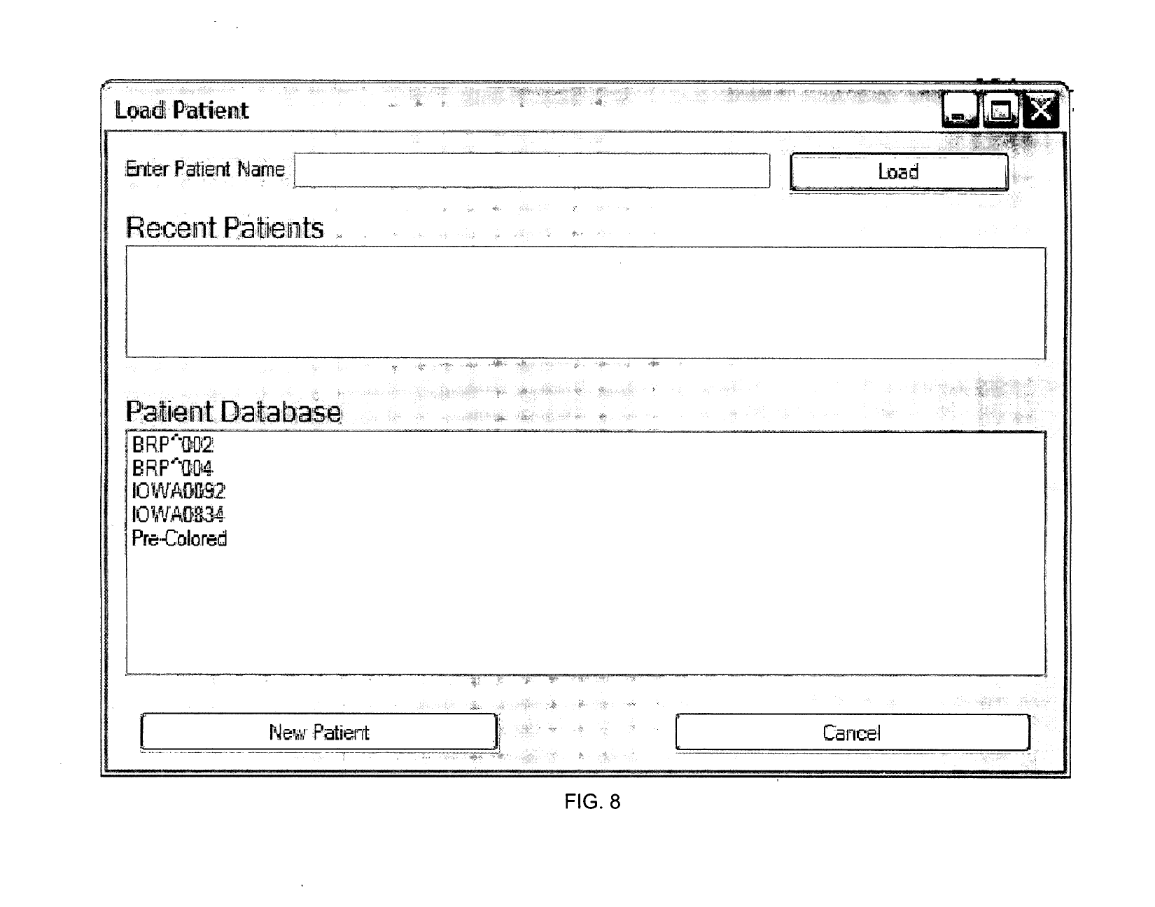

Pulmonary Workstation may be configured such that all the patient data and analysis it performs may be stored in a database or some other data storage device. Pulmonary Workstation may be configured such that choosing the “Review/Analyze Screen” link brings up a “Load Patient” dialog box, such as the one shown in FIG. 8. Pulmonary Workstation may be configured such that the patient data is organized by patient name and address. In the version shown in FIG. 8, anonymized data is used, with only the case identifier being displayed. Pulmonary Workstation may be configured such that when the patient is selected, the user presses the “Load” button or double clicks on the patient ID in the “Patient Database” window to get to the Analyze Study window (see FIG. 7).

Loading New Patients into the Database

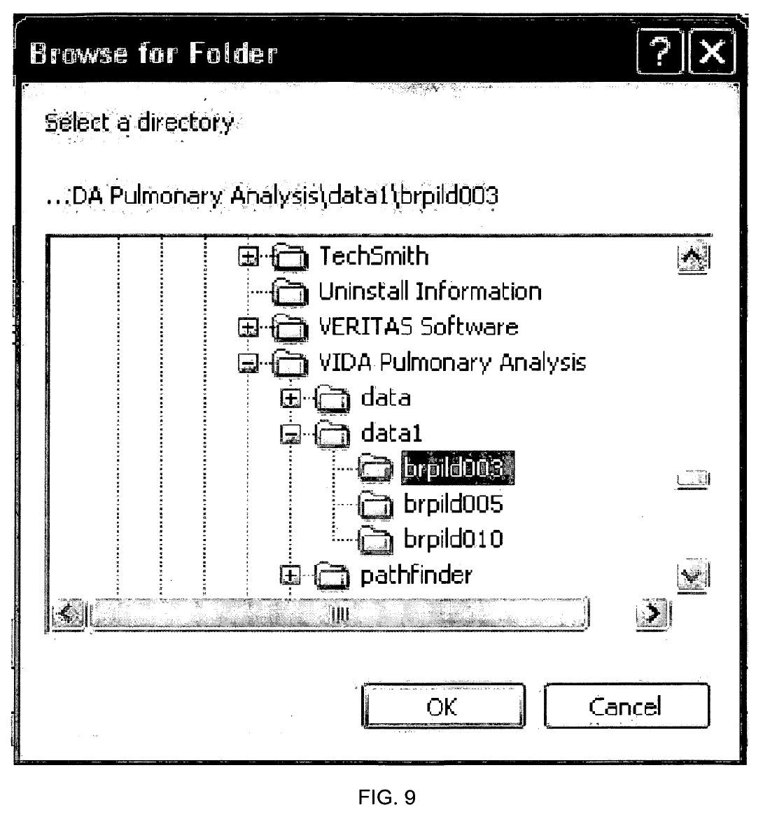

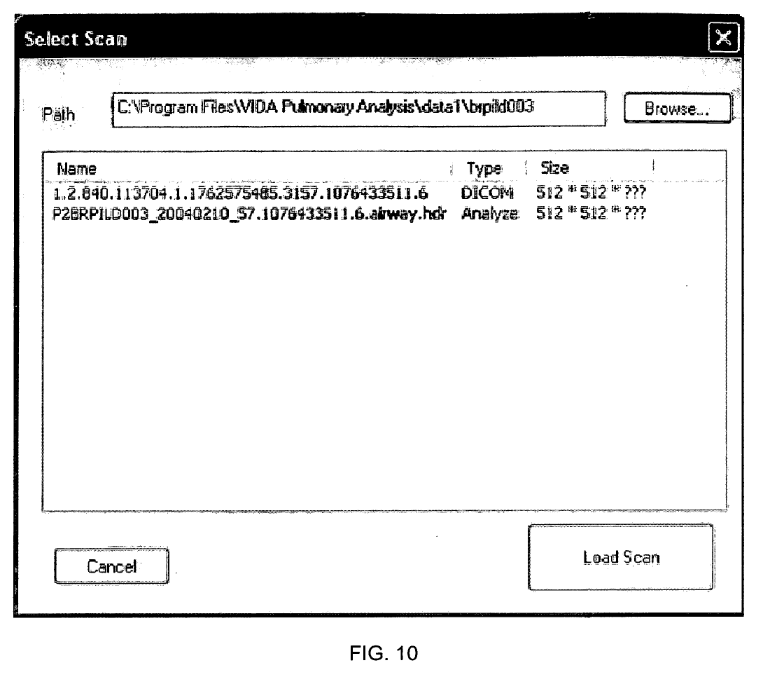

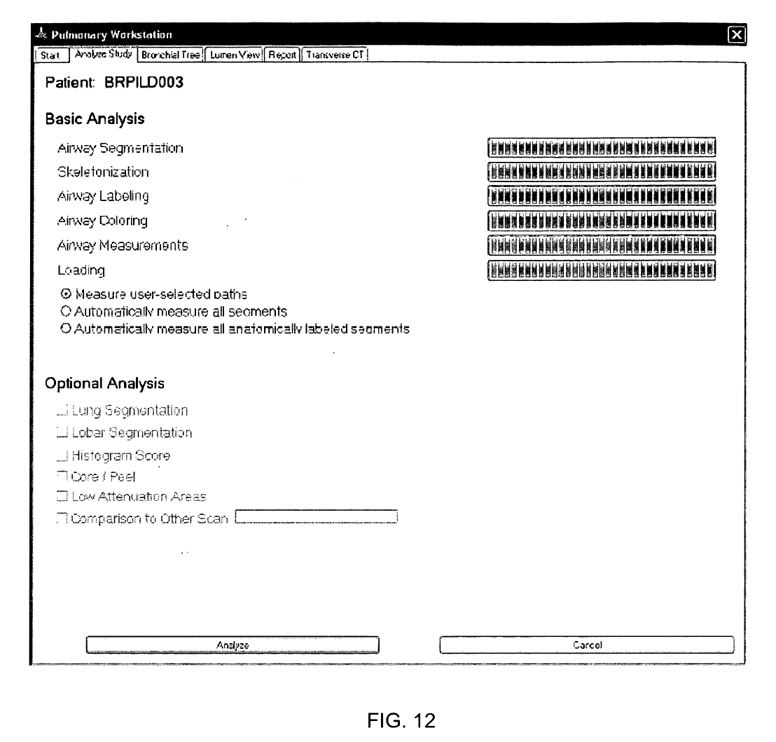

Pulmonary Workstation may be configured such that new patients/cases may be loaded by pressing the “New Patient” button in the lower left of the FIG. 8 screen. Pulmonary Workstation may be configured such that the New Patient button, when pressed, invokes the standard MS Windows® folder selection dialog box as seen in FIG. 9. The user may select the folder on a resident disk, removable disk or network drive as seen in the FIG. 9 dialog box, and click “OK” or double click on the selection. The application may then search that folder and only that folder for image files, which may be summarized by scan and displayed in the “Select Scan” dialog box shown in FIG. 10. Pulmonary Workstation may be configured such that highlighting a scan and double clicking it or clicking “Load Scan” causes that scan to be loaded into the application database. When the loading is complete, the user is advanced to the “Analyze Study” window shown in FIG. 12, where the user can determine what type of analysis to execute and then begin. The data that is loaded may be, for example, a CT volume from Analyze or DICOM files.

Patient Administration

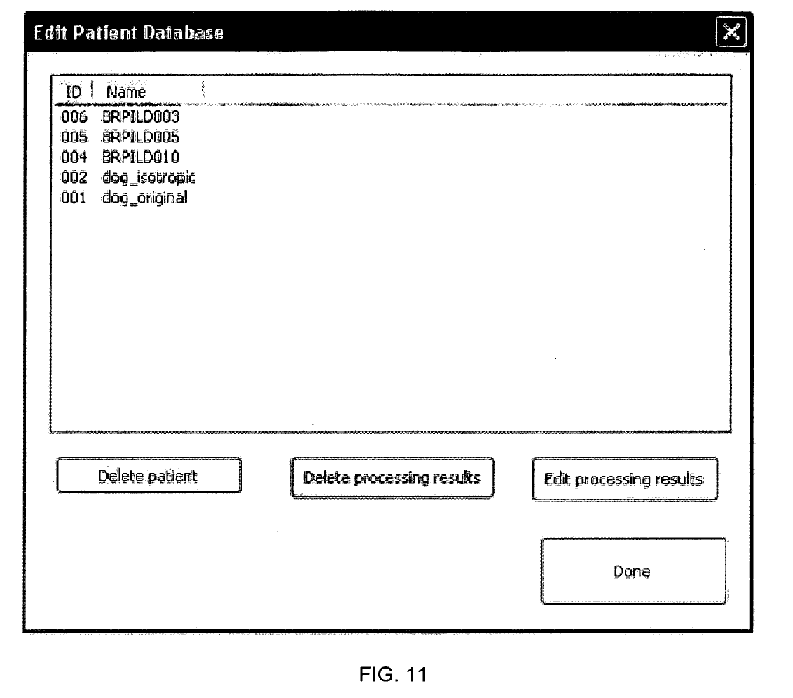

Pulmonary Workstation may be configured such that clicking the “Administer Patients” button on the main screen (FIG. 7) will bring up the window shown in FIG. 11, which may allow the user to manage patient data. Pulmonary Workstation may be configured such that the FIG. 11 window allows the user to delete a patient from the database, or to erase the analytical results for a patient. Pulmonary Workstation may be configured such that editing the analytical results allows the user to selectively delete analytical results, such as the measurements on an a la carte basis to enable new analysis with any updated software.

Airway Analysis

Pulmonary Workstation may be configured such that when the user selects a scan, the status of that scan is displayed in the “Analyze Study” window as shown in FIG. 12. Progress bars may show the status during and after the analysis. In the version shown in FIG. 12, the analysis has already been completed and automatically saved to the case database, so the progress bars are filled in on loading the case.

Pulmonary Workstation may be configured such that there are several options available for the airway measurement workflow. For example, Pulmonary Workstation may be configured such that if the user chooses the “Measure User Selected Paths” option (FIG. 12), airway measurements are done on the fly as the user selects the paths. Pulmonary Workstation may be configured such that the two automatic choices will cause the measurements to be made immediately after the airway segmentation, making the airway rendering/measurement run more quickly, but adding time to the initial analysis.

Pulmonary Workstation may be configured such that pressing the “Analyze” button shown in FIG. 12 (lower left) completes any remaining analysis and brings the user to the airway display (see the “Bronchial Tree” tab shown in FIG. 7).

The Bronchial Airway Tree Window

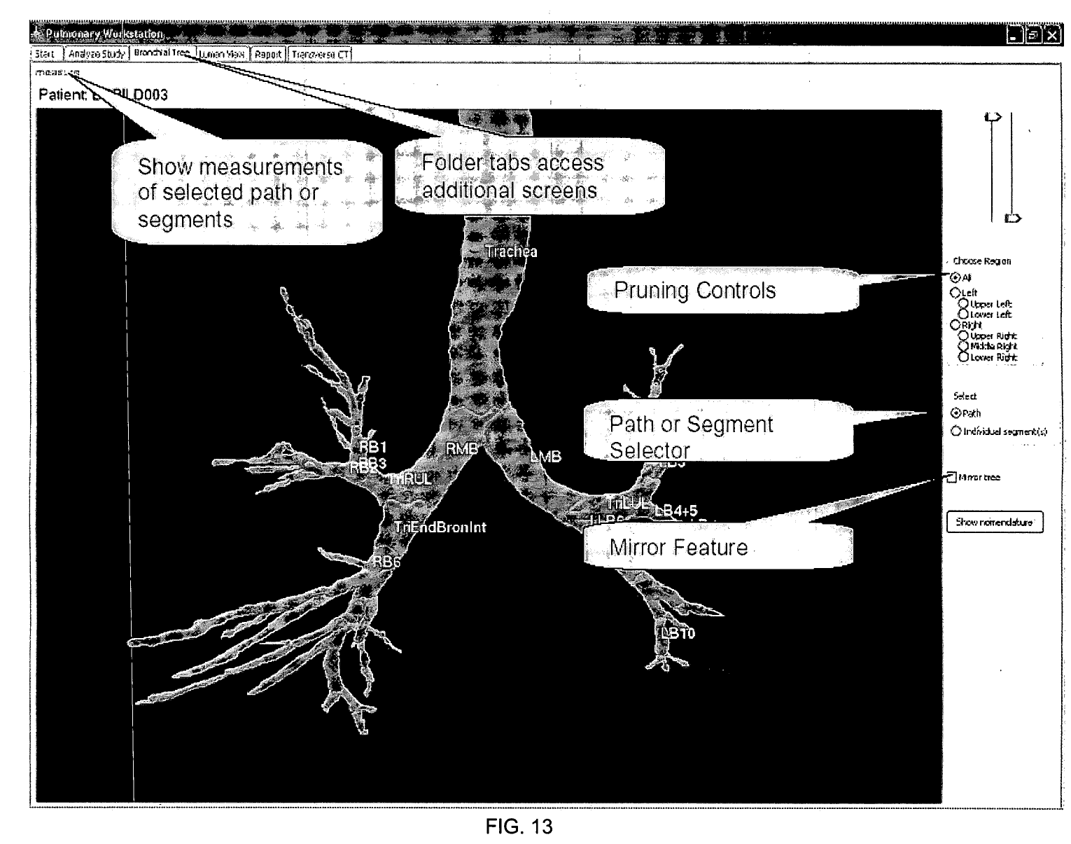

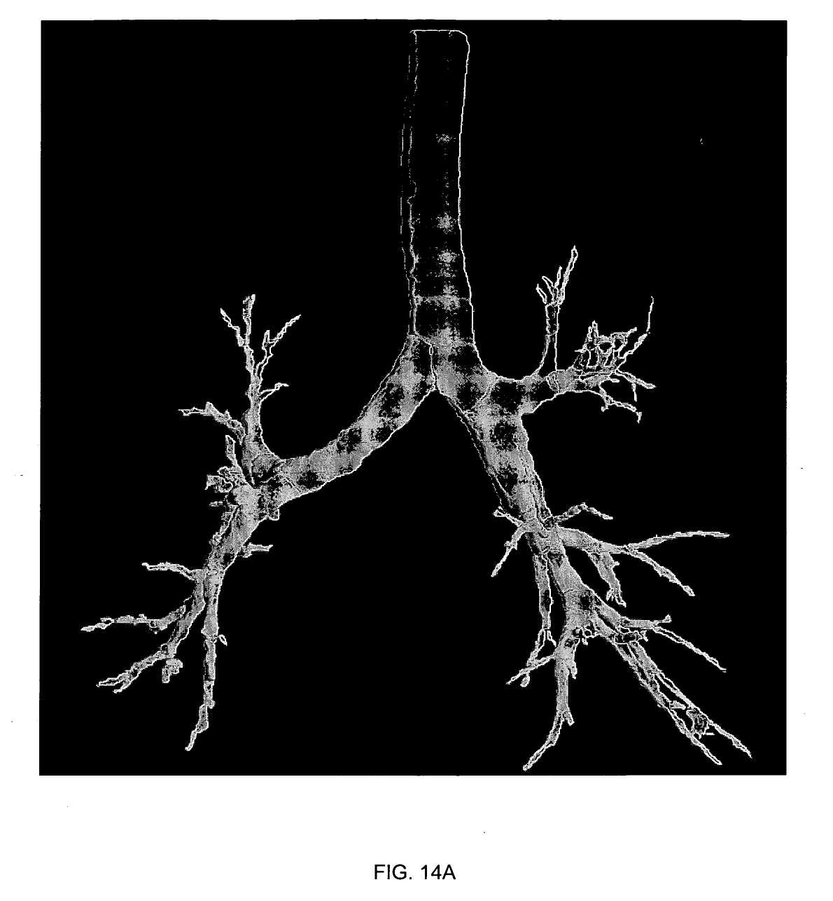

FIG. 13 shows that Pulmonary Workstation may be configured with a bronchial (or airway) tree window on which certain controls are positioned. As shown, Pulmonary Workstation may be configured such that the airway system is automatically divided into segments for the purpose of measurement.

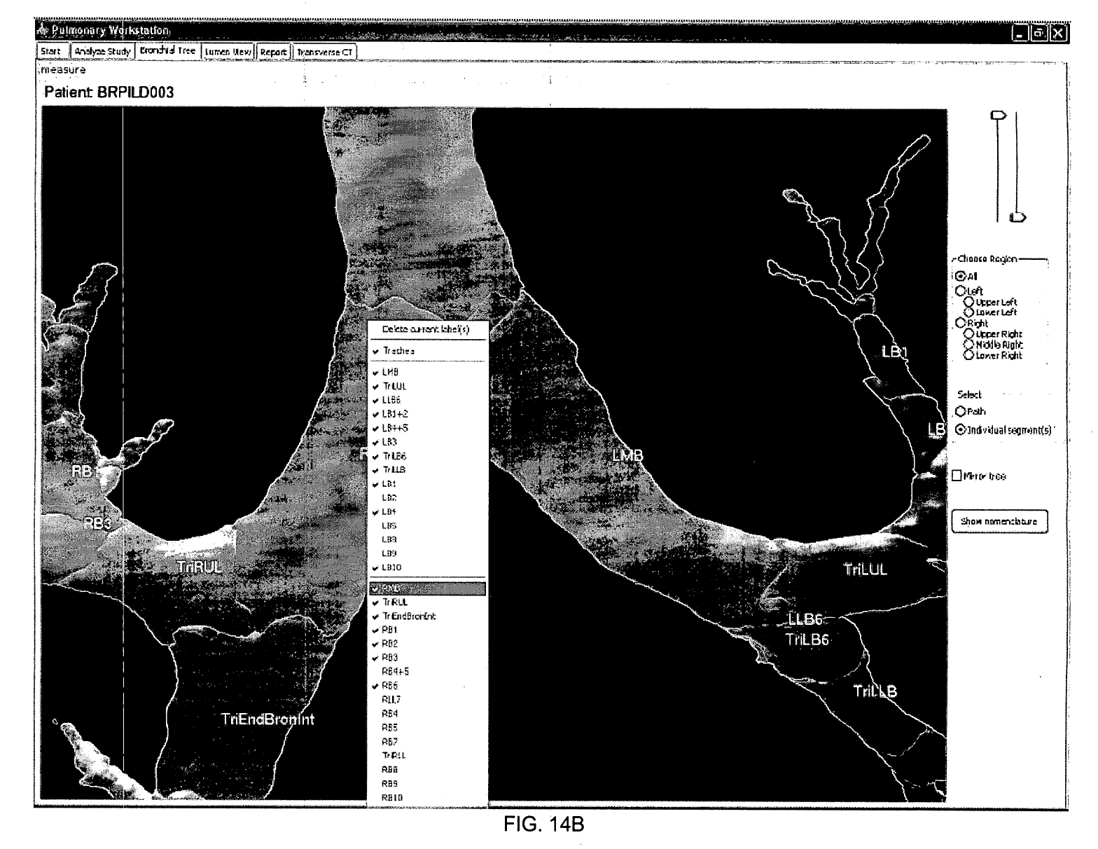

Pulmonary Workstation may be configured such that the following controls are available to the user:
1) left click and drag rotates the airway tree, both from side to side and from top to bottom;
2) right click and drag zooms in and out;
3) middle mouse click and drag causes the image to be relocated in the window, allowing, e.g., closer examination of a zoomed image;
4) double clicking on a segment of the airway tree selects a path for the Lumen View screen, which is the screen that may be configured to allow for lumen measurement. The path will be highlighted from the trachea to the segment that was double-clicked;
5) pruning controls: the pruning controls (labeled as such in FIG. 13) cause the tree to be truncated. The sliders will remove successive generations from the top to bottom (left slider) or from bottom to top (right slider). The “Choose Region” buttons below the sliders allow pruning by anatomical means, left or right lung or by lobe;
6) folder tabs: the folder tabs at the top of the page (see FIGS. 7, 12 and 13) allow for limited movement between screens;
7) path selection: when the “Select” radio button is set to “Path,” once a segment of the airway is selected by a double click, the tree display changes to highlight the path from the trachea through the selected segment (see FIG. 14A). Double clicking another segment will change the path to terminate at the newly chosen segment. When the “Select” radio button is set to “Individual segment(s),” double clicking a segment will highlight only that segment (see FIG. 14B). Control-double clicking an additional segment(s) in this mode will highlight multiple segments, whether they are in sequence or not. Although not shown in the figures, Pulmonary Workstation may be configured such that a user can select a path other than one that begins with the trachea. The functionality described here with respect to paths that begin with the trachea would apply with equal force to paths that begin downstream of the trachea.

Editing the Airway Nomenclature

Pulmonary Workstation may be configured such that once an individual airway segment is selected via a double click, a right click on that segment will bring up a menu for changing the airway's automatically selected name, as seen in FIG. 14B, and a user may select the new airway name desired. Pulmonary Workstation may be configured such that the downstream segments will be automatically renamed. Pulmonary Workstation may be configured such that if no change is desired, hitting the escape key (Esc) or clicking outside of the menu cancels the action.

Airway Measurements

Pulmonary Workstation may be configured such that once a path from the trachea to a selected segment is highlighted as described above, or when one or more individual segments are highlighted as described above, clicking the “measure” button on the upper left of the screen (see, e.g., FIG. 14B) moves the user to the “Lumen View” screen to examine the lumen and wall measurements of the selected path or airway segments.

Pulmonary Workstation may be configured such that if the measurements have not been done yet, the measurements are done dynamically for each path as the paths are examined in the “Lumen View” window; however, the results are stored in a database so that for each case, no measurements need be taken twice. Pulmonary Workstation may be configured such that each segment from the whole tree may be measured at once, as indicated in the radio buttons in the “Analyze Study” window shown in FIG. 12.

The Lumen Detail Screen

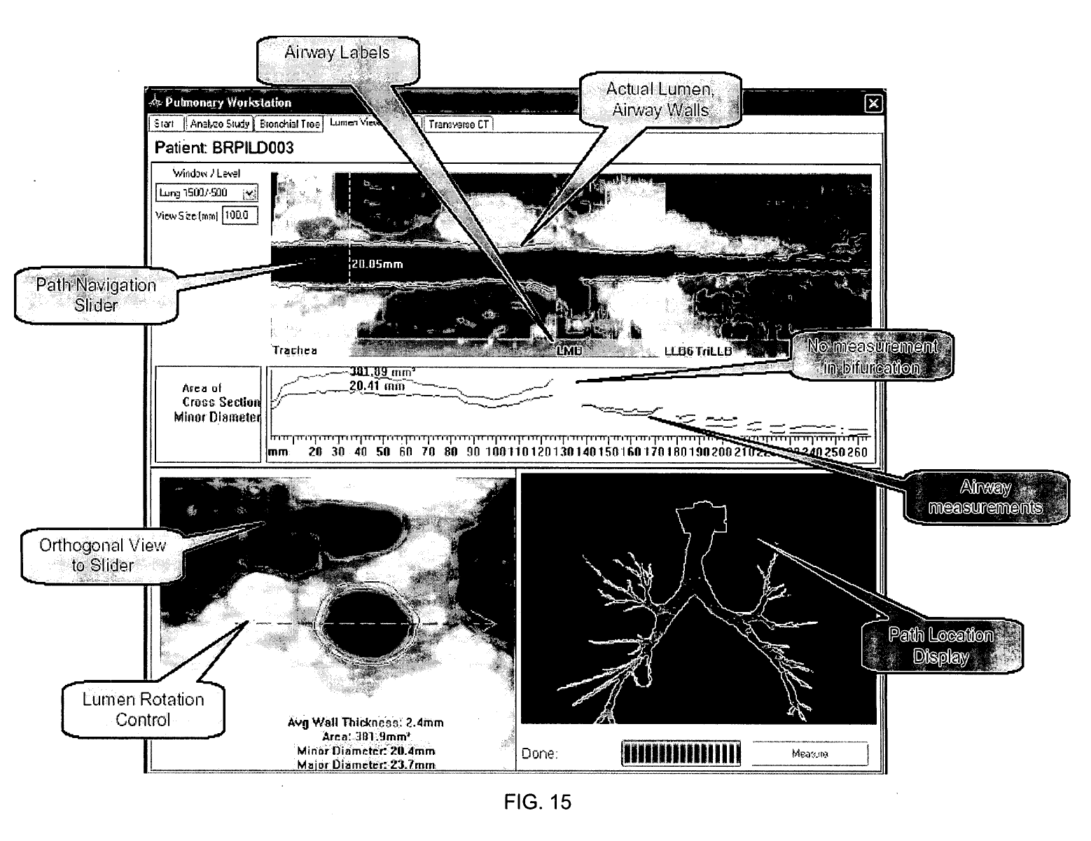

Pulmonary Workstation may be configured with the “Lumen View” screen depicted in FIG. 15. Pulmonary Workstation may be configured such that this screen appears instantly if the path chosen has been analyzed before. Pulmonary Workstation may be configured such that if a new path is chosen, or the path chosen is from a new case, the calculation could take up to 30 seconds.

Pulmonary Workstation may be configured such that the selected path, segment or group of segments (segments that are not aligned will appear in the FIG. 15 view in the order in which they have been selected) are straightened and shown in profile across the top of the screen shown in FIG. 15 with complete airway labeling.

Pulmonary Workstation may be configured such that the straightening comprises taking any curve or curves out of the segment, segments or path. This configuration may be achieved, and the resulting straightened section depicted, as follows. A centerline may be assigned to the path to be straightened (which, for this purpose may include only one segment). The centerline may be characterized as extending between the beginning (e.g., point A) of the path to be straightened and the end of the path to be straightened (e.g., point B). The centerline may be divided into small steps. The small steps may be defined by the closest image voxels. However, any other subdivision of the centerline may be used. The centerline may be divided into even or uneven steps. At each step location along the centerline, the gray-values of the original image volume are sampled along a line L that is positioned such that it runs perpendicular to the tangent at the current centerline position. The tangent may be determined by taking the first derivative of the spline representation of the centerline. The rotational position of line L (e.g., sample line L₀) and point A may be determined by the user (the rotational position may be defined by an angle in the range of between 0 and 360 degrees). Each subsequent sample line L₁, L₂, . . . , Lₙ may be positioned such that the angle between two neighboring sample lines Lₙ and Lₙ₊₁ is minimized. The gray-values sampled from the image volume along the lines L may be visualized in the longitudinal view shown in the profile window near the top FIG. 15. L₀ may be plotted in the left-most column of that profile view. L₁ may be plotted in the next column to the right, and so on. Lₙ, corresponding to the sample line at point B, may be plotted in the right most column of the FIG. 15 profile view. Changing the rotational position of L₀ allows the user to rotate the longitudinal view around the axis represented by the straightened centerline.

Pulmonary Workstation may be configured such that the outlines in red around the straightened portion of the airway tree represent the inner and outer walls of that portion, as determined according to the techniques discussed above in the Measurements of Airway Tree Geometry section.

Pulmonary Workstation may be configured such that the position of the viewing plane for the straightened path is seen in the lower left panel, which is an orthogonal view of the airway lumen and walls. Pulmonary Workstation may be configured such that the green arrow that bisects the orthogonal view shows the orientation of the path cross-section shown in the top FIG. 15 window. Pulmonary Workstation may be configured such that grabbing and dragging this arrow will rotate the path and adjacent CT view (or view from whatever suitable imaging modality is used). Pulmonary Workstation may be configured such that when the “Lumen View” folder is clicked, the position of this arrow is reset. Pulmonary Workstation may be configured such that the profile of the inner and outer walls of an airway segment at the location of the “Path Navigation Slider” discussed below are overlaid in color (e.g., red) on the orthogonal image depicted in the lower left window of the “Lumen View” screen (as shown in FIG. 15). Pulmonary Workstation may be configured such that certain measurements are provided graphically in the lower left window corresponding to the particular portion of the airway segment being depicted, such as the “average wall thickness” (e.g., the mean wall thickness), the cross-sectional area determined using the border of the inner airway wall of the designated location of the airway segment, the minor diameter (e.g., the inner diameter) of the designated location of the airway segment, and the major diameter (e.g., the outer diameter of the exterior of the airway segment) of the designated location of the airway segment.

Pulmonary Workstation may be configured such that the orthogonal view in the lower left of FIG. 15 is controlled by the yellow dashed line labeled “Path Navigation Slider” that can be slid across the profile (from left to right) to navigate the orthogonal view. This dashed line is an example of a “location indicator”. Pulmonary Workstation may be configured such that when the orthogonal view has been clicked, the slider will be reset to a beginning position, such as by returning the orthogonal view across the path it traversed by virtue of moving the slider. Pulmonary Workstation may be configured such that the number in yellow next to the dotted slider represents the inner airway diameter at that particular position. Pulmonary Workstation may also be configured to yield any other measurement at that position (e.g., cross-sectional area (based on the inner airway diameter), outer wall diameter, wall thickness, etc.

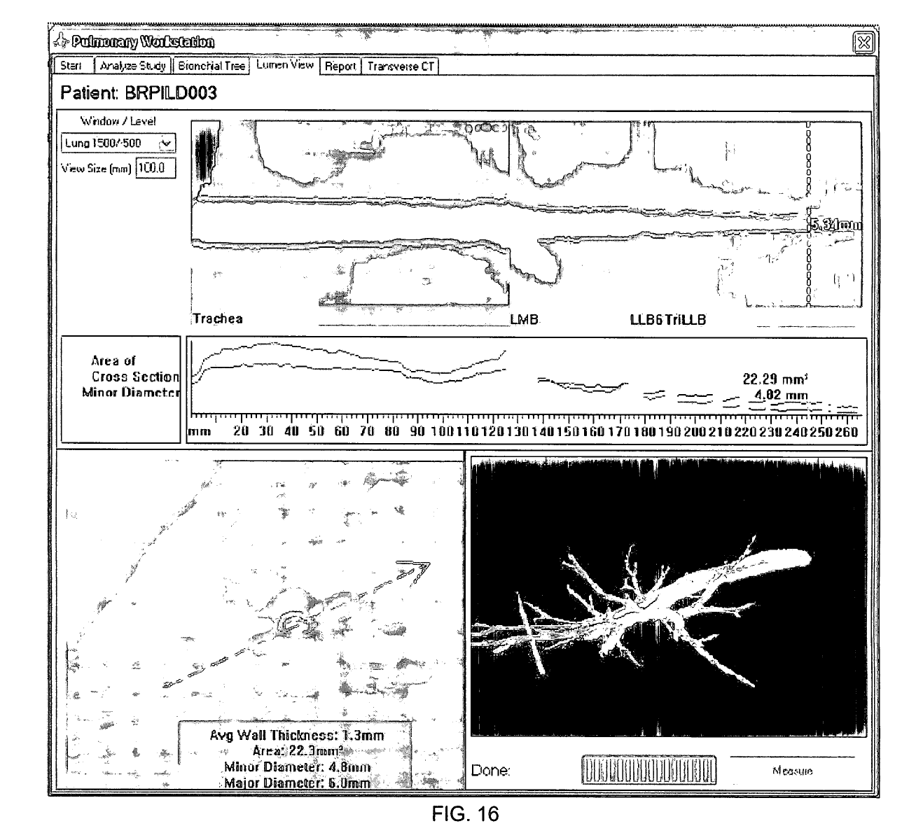

Pulmonary Workstation may be configured such that the Path Location Display (labeled as such in FIG. 15) shows the position of the orthogonal view in 3D space. Pulmonary Workstation may be configured such that the bronchial tree view in this window may be manipulated with the same rotate, zoom and move functions in the main “Bronchial Tree” window. Pulmonary Workstation may be configured such that in the example shown in FIG. 16, the path location has been moved to near the distal end of the path. Pulmonary Workstation may be configured such that the “position plane” shown in the Path Location Display window (see FIGS. 15 and 16) is located at the same position as the slider shown in the profile window.

Pulmonary Workstation may be configured such that the “Lumen View” screen includes an airway measurements window beneath the profile window (see FIG. 15). Pulmonary Workstation may be configured to display a scale corresponding to the straightened length of airway segment(s) shown in the profile window, as shown in FIG. 15. Pulmonary Workstation may be configured such that the path navigation slider overlaps both the profile and airway measurement windows, such that movement of the slider can be linked to a specific location on the scale. Pulmonary Workstation may be configured such that at least some of the measurements determined according to the Measurements of Airway Tree Geometry section above are displayed graphically in the airway measurements window. For example, Pulmonary Workstation may be configured such that minor diameter (which, in the case of an airway segment, is the inner diameter at a given location along the segment (specifically, the location intersected by the slider, in this embodiment)) is depicted in one color (blue, in this embodiment) and the cross-sectional area of the interior (or the exterior) of the airway segment at a given location along the segment (specifically, the location intersected by the slider, in this embodiment) is depicted in another color (red, in this embodiment). Pulmonary Workstation may be configured such that these measurements are available along some or all of the portion of the airway segment depicted in a straightened form in the profile window except for bifurcation(s) of the segment in question.

Pulmonary Workstation may be configured such that if an additional or different airway segment or path needs to be examined, a user can (using the folder tabs at the top of the screen) return to the “Bronchial Tree” page and select a new path/segment(s), using the technique described above.

Pulmonary Workstation may be configured such that the “Window/Level” settings of the gray-level images may be changed, as shown in the upper left-hand corner of the FIGS. 15 and 16 screens.

Saving the Data to Another Format

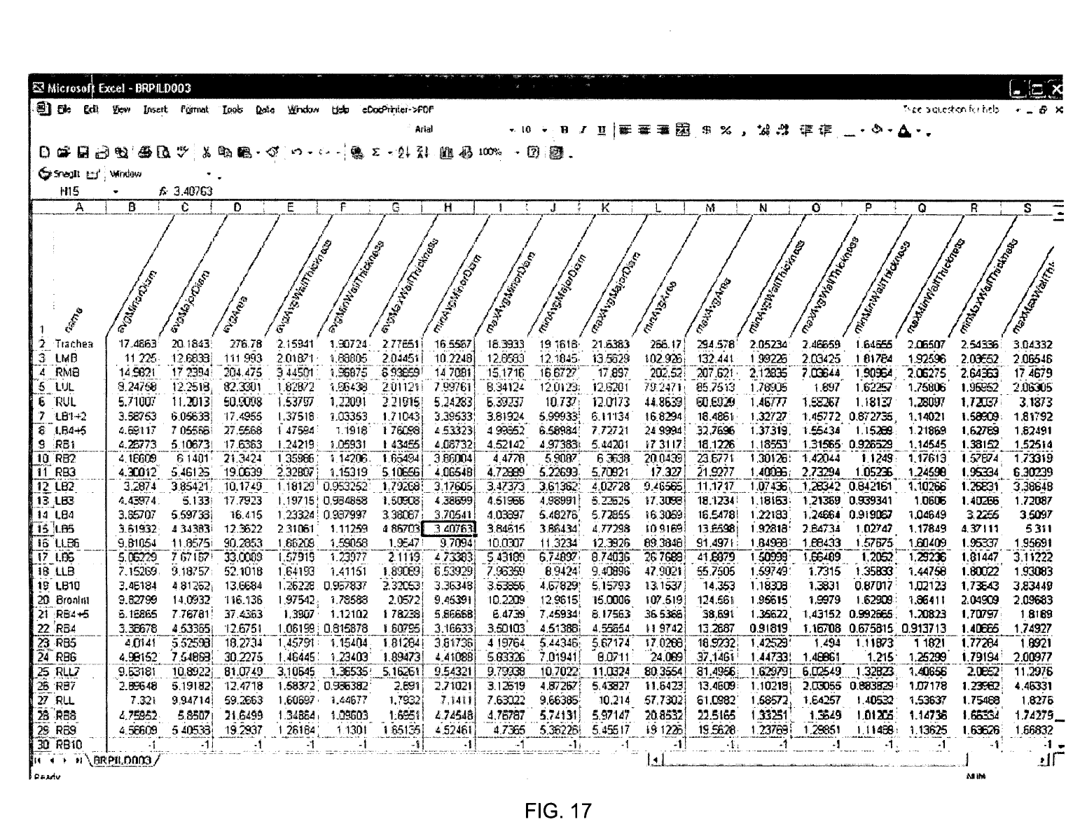

Pulmonary Workstation may be configured such that clicking the folder tab labeled “Report” allows the measurement data that has been determined to be outputted to, for example, a CVS format text file. Pulmonary Workstation may be configured such that the default setting creates a space (ASCII 32) delimited file, while other delimiters can be chosen. Pulmonary Workstation may be configured such that when loaded into Microsoft® Excel, the file will trigger the autoparse function, which allows the user to choose “delimited” parsing, which arranges the columns based on the delimiter character chosen. Pulmonary Workstation may be configured such that the resulting spreadsheet, with some formatting of the labels, looks like the spreadsheet depicted in FIG. 17. Pulmonary Workstation may be configured such that only the segment(s) that have been measured will be output in this fashion. Pulmonary Workstation may be configured such that a “−1” value is inserted into a row corresponding to a segment that has not been measured (see RB10 row of FIG. 17).

As FIG. 17 shows, Pulmonary Workstation may be configured such that the following parameters are determined for a given segment:

average minor diameter (labeled as “avgMinorDiam”), which comprises the arithmetic average of individual minor diameter measurements conducted at different locations along the segment;

average major diameter (labeled as “avgMajorDiam”), which comprises the arithmetic average of individual major diameter measurements conducted at different locations along the segment;

average cross-sectional area (labeled as “avgArea”), which comprises the arithmetic average of individual cross-sectional area measurements conducted at different locations along the segment;

average value of average wall thickness (labeled as “avgAvgWallThickness”), which comprises the arithmetic average of individual average wall thickness measurements conducted at different locations along the segment;

average minimum wall thickness (labeled as “avgMinWallThickness”), which comprises the arithmetic average of individual minimum wall thickness measurements conducted at different locations along the segment;

average maximum wall thickness (labeled as “avgMaxWallThickness”), which comprises the arithmetic average of individual maximal wall thickness measurements conducted at different locations along the segment;

minimum average minor diameter (labeled as “minAvgMinorDiam”), which comprises the minimum value of individual average minor diameter measurements conducted at different locations along the segment;

minimum average major diameter (labeled as “minAvgMajorDiam”), which comprises the minimum value of individual average major diameter measurements conducted at different locations along the segment;

minimum average cross-sectional area (labeled as “minAvgArea”), which comprises the minimum value of individual average cross-sectional area measurements conducted at different locations along the segment;

maximum average cross-sectional area (labeled as “maxAvgArea”), which comprises the maximum value of individual average cross-sectional area measurements conducted at different locations along the segment;

minimum average wall thickness (labeled as “minAvgWallThickness”), which comprises the minimum value of individual average wall thickness measurements conducted at different locations along the segment;

maximum average wall thickness (labeled as “maxAvgWallThickness”), which comprises the maximum value of individual average wall thickness measurements conducted at different locations along the segment;

minimum value of minimum wall thickness (labeled as “minMinWallThickness”), which comprises the minimum value of individual minimal wall thickness measurements conducted at different locations along the segment;

maximum value of minimum wall thickness (labeled as “maxMinWallThickness”), which comprises the maximum value of individual minimal wall thickness measurements conducted at different locations along the segment;

minimum value of maximum wall thickness (labeled as “minMaxWallThickness”), which comprises the minimum value of individual maximal wall thickness measurements conducted at different locations along the segment; and

maximum value of maximum wall thickness (labeled as “maxMaxWallThickness”), which comprises the maximum value of individual maximal wall thickness measurements conducted at different locations along the segment.

The preceding parameters are examples of dimensional attributes of a given selected portion of an airway tree.

Another example software package that may be used to practice some embodiments of the steps described above will be referred to as “Pathfinder” in this disclosure. Those of ordinary skill in the art having the benefit of this disclosure will be able to write code (machine readable instructions) without undue experimentation for accomplishing the features of Pathfinder (including the graphical user interface) described below and shown in the figures.

### Pathfinder

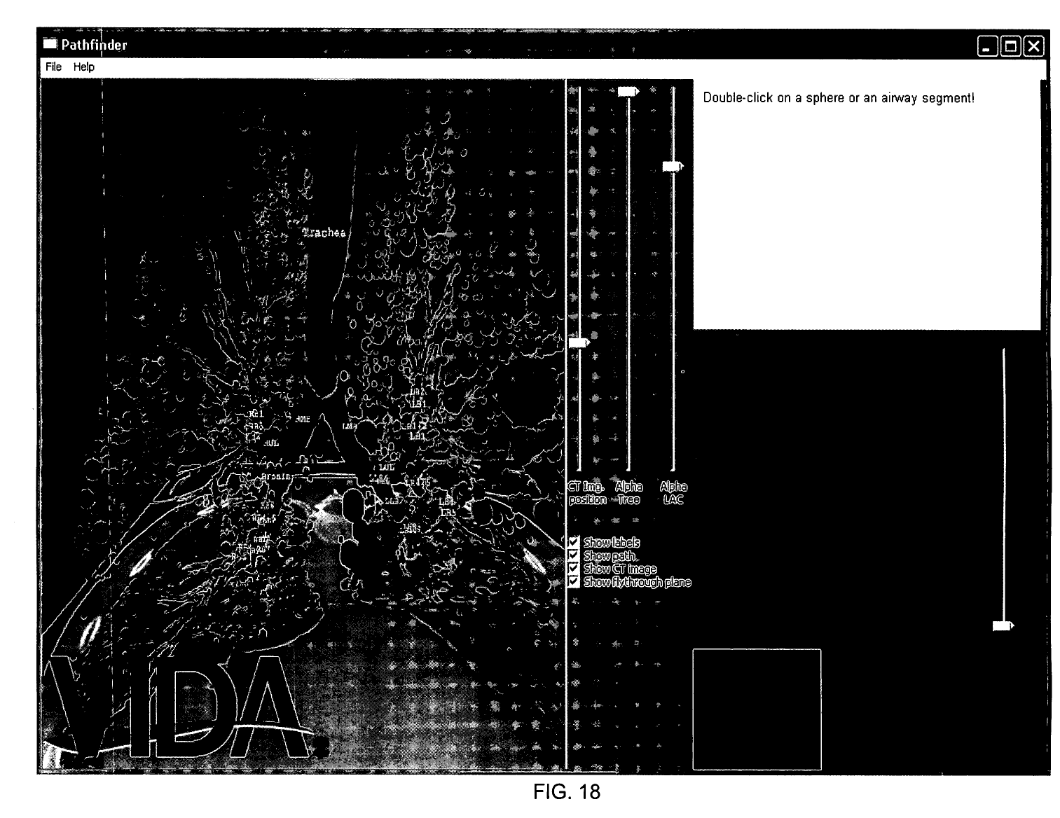

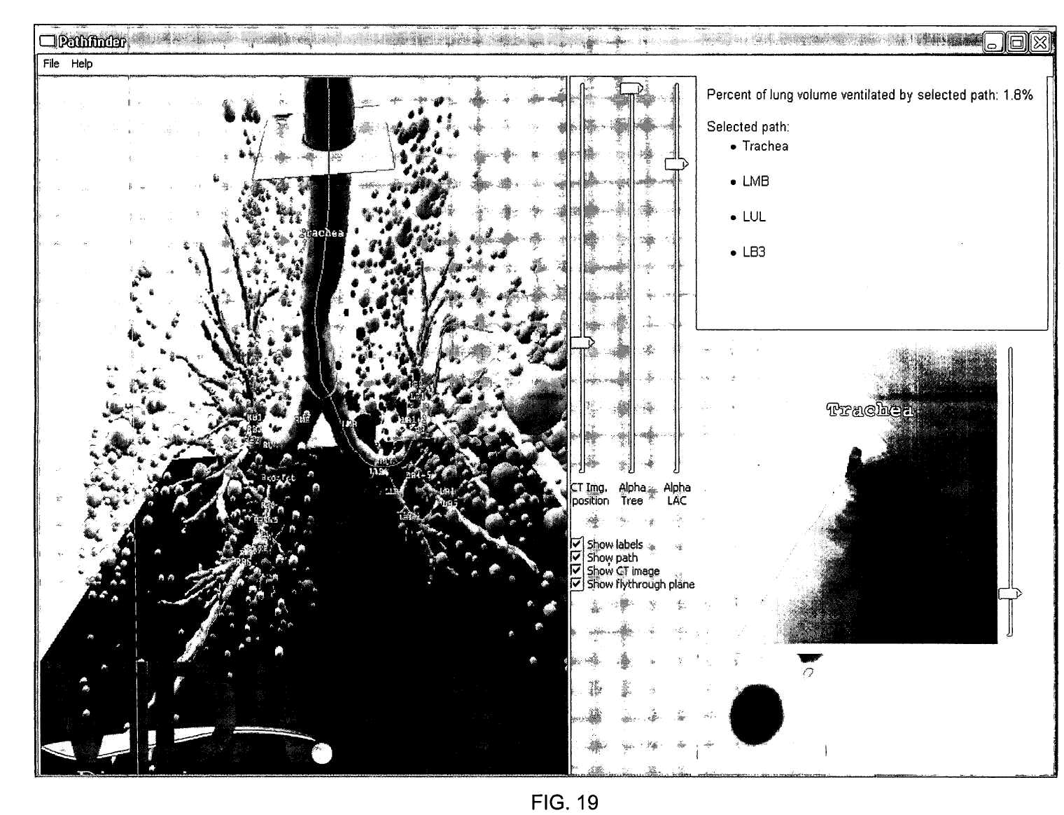

Pathfinder may be configured to produce the graphical user interface (GUI) shown in FIGS. 18-21. The GUI shown is configured for the treatment planning of emphysema. However, Pathfinder (as well as Pulmonary Workstation) may be configured for the treatment planning of any intervention that involves a body lumen and concerns any disease or condition of interest.

As FIGS. 18-21 show, Pathfinder may be configured to present a window showing a segmented, skeletonized and anatomically-labeled bronchial tree (created according to, for example, the techniques disclosed above) together with LACs that are identified and clustered according to, for example, the techniques disclosed above. Pathfinder may be configured so that LACs within the same lobe have the same color, as shown in these figures. A given sphere represents an air cluster indicative of emphysema. Pathfinder may be configured such that the “CT Image position” slider next to the segmented bronchial tree window may be moved, and the image from the volumetric dataset of images defining the tissue of interest (e.g., a CT slice that is part of the dataset of CT images depicting a given set of lungs) may be superimposed on the relevant portion of the bronchial tree. Pathfinder also may be configured such that the “Alpha Tree” slider controls the intensity of the depiction of the segmented bronchial tree (thus, moving the Alpha Tree slider all the way to one end of the range makes the bronchial tree invisible), and the “Alpha LAC” slider controls the intensity of the depiction of the LACs (the differently colored spheres) such that moving the Alpha LAC slider all the way to one end of the range makes the LACs invisible.

Pathfinder may be configured such that the bronchial tree may be manipulated in a similar or identical manner to the way in which the bronchial tree depiction in the “Bronchial Tree” screen of Pulmonary Workstation may be manipulated. Thus, and as an example, Pathfinder may be configured such that:

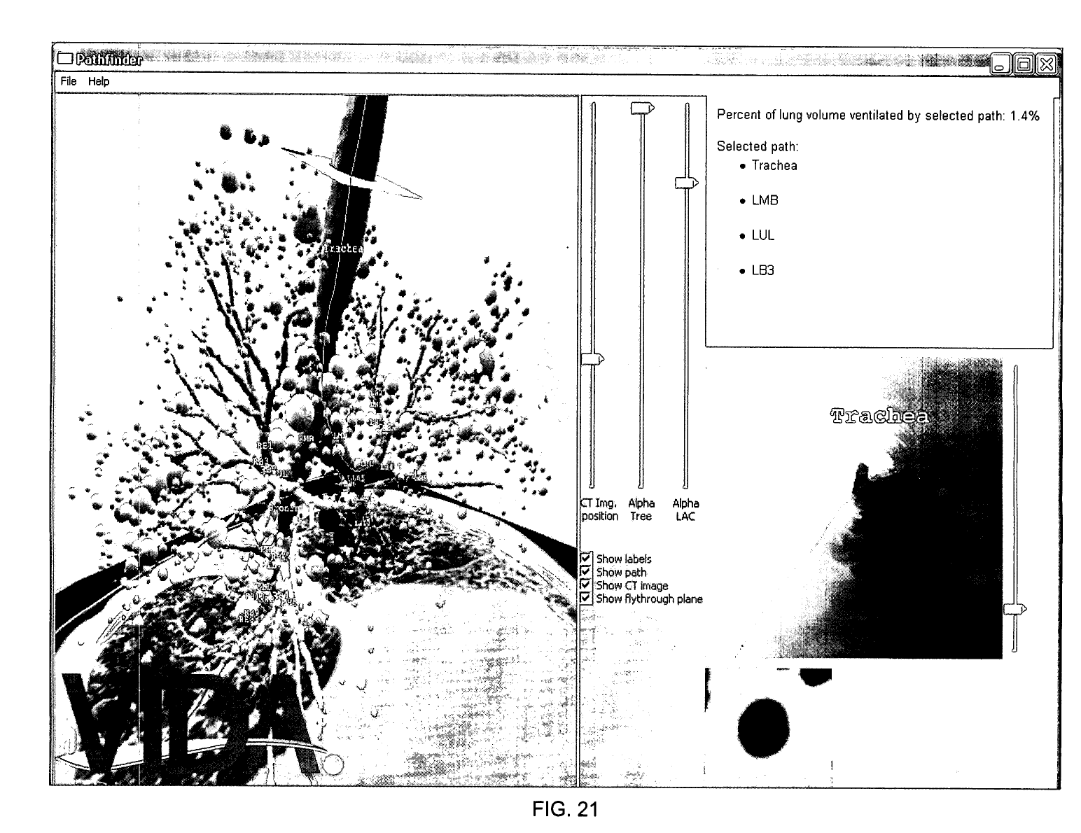

dragging a mouse with the left mouse key held down rotates the tree (FIG. 21 shows a tree that has been rotated);

dragging the mouse with the middle mouse key held down (or, e.g., a roller wheel) translates the position of tree; and

dragging the mouse with the right mouse key held down zooms.

Pathfinder may be configured such that double-clicking on a sphere causes the pathway through the bronchial tree to a location within the bronchial tree that is closest to that sphere to be determined (e.g., computed) according, for example, to the techniques discussed above in the Identifying Relevant Airway Segments section and displays that pathway, such as with a red line as shown in FIGS. 19-21 (which is one example of displaying one or more locations within a portion of a set of lungs that are therapeutically linked to a target treatment region) and/or with a description of the labeled segments making up the path, as shown in the upper right-hand window in FIG. 19 designated by “Selected path”. Pathfinder may be configured such that the double-clicked sphere is highlighted (the highlighted spheres in the figures are those that are not shaded), as are the spheres that are fed by the same path that feeds the selected sphere, as shown in FIGS. 19 and 21 (two different spheres were originally clicked for these figures, as evidenced by the red line tied to different spheres). Pathfinder may be configured such that, for a selected path, the percentage of lung volume ventilated by the selected path may be determined and displayed (e.g., see the “Percent of lung volume ventilated by selected path” designation in the upper right-hand window in FIG. 19). Pathfinder may be configured such that the percentage of lung volume ventilated by a selected path is computed as follows: for every terminal airway segment, the number of (non-airway) lung voxels within the same lobe that are geometrically closer to that terminal segment than to any other terminal segment within the same lobe are determined. This count, compared to the total count of voxels within the lungs, yields the numerical value of the percent of lung volume ventilated by the respective terminal segment. Because every “feeding airway path” is leading through a terminal airway segment, the “percent of ventilated lung volume” number can be determined through the corresponding terminal airway segment.

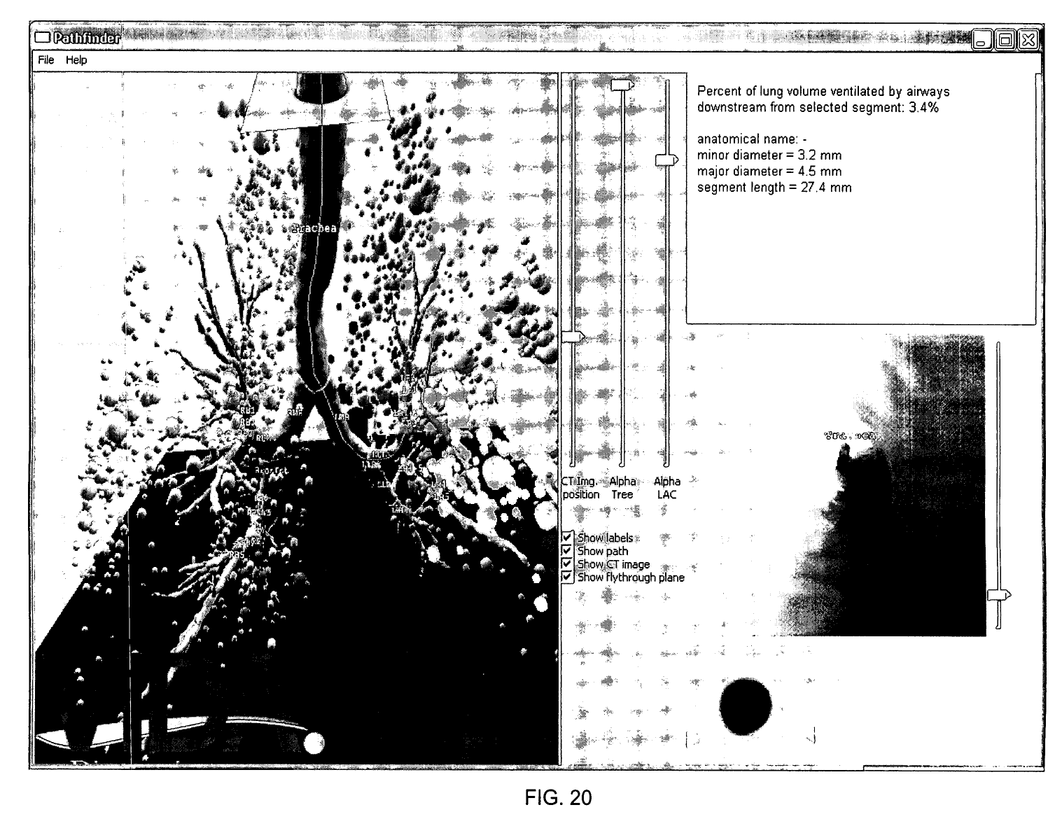

Pathfinder may be configured such that double-clicking on an airway segment effectively constitutes placing a one-way valve (or some other therapeutic device, such as a stent) in that segment because, in some embodiments, it triggers a determination of the LAC or LACs that are downstream of (or fed by) the double-clicked segment, and highlights only those spheres (such that the target sphere may be de-highlighted if it is not one of those downstream LACs, as in FIG. 20; such highlighting is one example of indicating that a target treatment region is therapeutically linked to a selected location for a therapeutic device); the selected segment is also highlighted (e.g., in red, as shown in FIG. 20). Pathfinder may be configured such that this determination may be made as described above in the section entitled Identifying Relevant Airway Segments. If a user determines that the target sphere is not among those highlighted when a given airway segment is selected for mock placement of a therapeutic device such as an airway valve, Pathfinder may be configured such that double-clicking on another segment will trigger the same determination, such that the user may iteratively keep testing segments until those spheres that are highlighted as a result include the target sphere. Pathfinder may be configured such that once a target sphere is selected by, e.g., double-clicking on that sphere, segments that are tested (e.g., by double-clicking on them) and that do not feed the target sphere may be designated as such (e.g., by making a different color from the other segments, such as gray) so that a user does not mistakenly test them again in the process of finding a suitable feeding airway segment.

Pathfinder also may be configured such that when a given airway segment is double-clicked, the percentage of lung volume that is ventilated by the selected airway segment (e.g., the portion of the airway tree that is downstream from the selected segment) may be determined (e.g., computed) and displayed in another window (see the “Percent of lung volume ventilated by airways downstream from selected segment” designation in the upper right-hand window of FIG. 20). Pathfinder may be configured such that this percent number is computed by first identifying the terminal airway segments that are situated topologically underneath (downstream) from the selected segment. Every such terminal segment has a “percent of ventilated lung” number that may be associated with it, as described above. The sum of these percent numbers represents the total percentage of lung volume ventilated by the selected segment. Pathfinder also may be configured to display the anatomical name of the selected segment and any one or more of the measurements discussed above for a selected segment, as shown in the upper right-hand window of FIG. 20.

As FIGS. 19-21 show, Pathfinder may be configured to display a “fly-through” window that allows a user to perform a virtual bronchoscopy along a path selected as described above using the depicted slider, which controls the position of the “position plane” shown in the airway tree window to the left. Techniques for virtual bronchoscopy are well known in the art, and, accordingly, will not be described further here. Pathfinder may be configured to display the anatomical label of the segment being navigated in the virtual bronchoscopy window (see FIGS. 20 and 21). Pathfinder may be configured to display an image (e.g., a resampled CT image) in a window next to the fly-through window that corresponds to the position of the “position plane” shown in the bronchial tree window, and thus to the current position of the virtual bronchoscope. This allows the user, for example, to determine the position of vessels outside the airway wall, which may be important information for the planning of, for example, a needle biopsy.

### Validation

Lung Segmentation: Low-dose datasets (120 kV, 50 mAs) from 10 different emphysema patients were available. The resolution of the scans was 0.66×0.66×0.6 mm³. A human expert hand-traced the lung borders in 6 randomly selected axial slices. For each border point in the automatically-obtained lung segmentation result, the closest point in the reference dataset was determined and the 3D Euclidean distance between each such pair of points was measured. Table 1 summarizes the results of the error measurements, showing all values in millimeters:

TABLE 1
Error measurements in lung segmentation result.

| | RMS Error | | |
|---|---|---|---|
| Subject | Left | Right | Combined |
| brpild003tlc | 0.61 | 0.53 | 0.56 |
| brpild005tlc | 0.91 | 1.21 | 1.15 |
| brpild008tlc | 2.74 | 1.70 | 2.50 |
| brpild009tlc | 0.47 | 0.48 | 0.47 |
| brpild010tlc | 0.41 | 0.45 | 0.43 |
| p2brp006tlc | 0.50 | 0.49 | 0.50 |
| p2brp044tlc | 0.52 | 0.61 | 0.58 |
| p2brp048tlc | 0.43 | 0.43 | 0.43 |
| p2brp054tlc | 0.54 | 0.90 | 0.79 |
| p2brp055tlc | 0.45 | 0.48 | 0.46 |
| Average | 0.76 | 0.73 | 0.79 |

Table 1 shows the root mean squared (RMS) errors for the left, right, and both lungs; the signed errors and unsigned errors showed similar results. In all cases the signed error is relatively close to zero and well below the CT scanner resolution, which indicates that no significant systematic error is present.

Lobe Segmentation: Low-dose datasets (120 kV, 50 mAs) from 10 different emphysema patients were available. The resolution of the scans was 0.66×0.66×0.6 mm³. A human expert hand-traced each of the 3 fissures (left oblique, right oblique, right horizontal) in 6 randomly selected sagittal slices. For each fissure point in the automatically obtained lobe segmentation result, the closest point in the reference dataset was determined and the 3D Euclidean distance between each such pair of points was measured. Table 2 summarizes the results of the error measurements.

TABLE 2
Error measurements in lobe segmentation result. Measuring
location of fissure lines between lobes. All values are in millimeters.
Missing values due to failed segmentation are designated with a ‘—’.

| | RMS Error | | |
|---|---|---|---|
| | Left | Right | Right |
| Subject | Oblique | Oblique | Horizontal |
| brpild003tlc | 2.00 | 1.88 | 1.30 |
| brpild005tlc | — | 1.85 | 3.71 |
| brpild008tlc | 2.63 | 4.02 | — |
| brpild009tlc | 1.47 | 2.88 | 3.10 |
| brpild010tlc | 6.26 | 2.23 | 4.60 |
| p2brp006tlc | 3.71 | 1.98 | 1.37 |
| p2brp044tlc | 1.00 | 1.70 | 2.27 |
| p2brp048tlc | 1.45 | 3.72 | 1.76 |
| p2brp054tlc | 2.33 | 1.31 | 1.90 |
| p2brp055tlc | 3.35 | 8.57 | 2.39 |
| Average | 2.47 | 2.64 | 1.93 |

The root mean squared (RMS) errors is listed separately for the left oblique fissure, the right oblique fissure, and the right horizontal fissure. Results for the signed errors and unsigned errors were similar. The detection of the left oblique fissure in subject brpild005tlc and the right horizontal in subject brpild008tlc failed because of badly deteriorated fissure lines (due to disease). For the two oblique fissures, the signed error is relatively close to zero, which indicates that no significant systematic error is present. The signed error for the right horizontal fissure is bigger because the right horizontal fissure mostly lies within the scan plane and due to that it exhibits a relatively low contrast. Also the scanner resolution is coarser in scan-direction, which additionally contributes to this increased error.

LAC Analysis

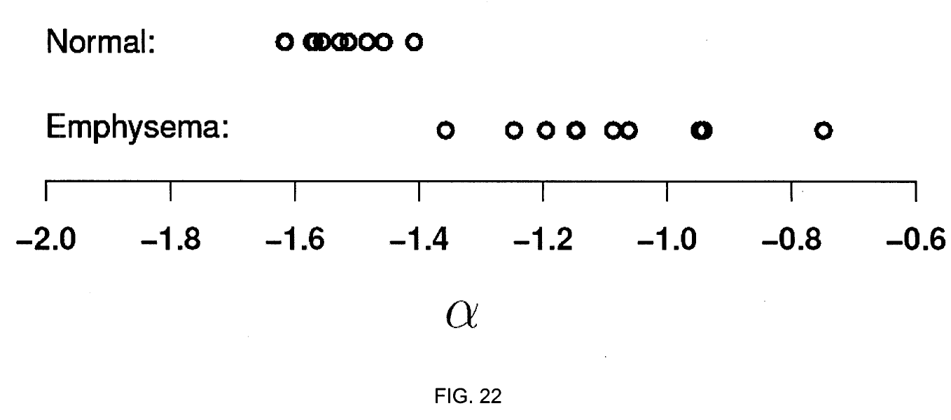

Scan from emphysema subjects and non-emphysema (normal) subjects were analyzed and the values for the slope α from the LAC log-log-plots between these two groups were compared. A total of 11 scans from emphysema subjects and 10 scans from normal subjects were available. FIG. 22 shows the results. With p=1.65 10⁻¹¹, the separation of the α values of the two groups is statistically highly significant.

Anatomical Labeling: Anatomical labeling was validated on a total of 10 normal subjects and 7 diseased subjects. A human expert provided a gold-standard by hand-labeling all 17 trees. On average, 27 labels were assigned correctly per tree. In contrast, an average of 0.82 labels were wrong per tree. This amounts to 97.1% correct labels vs. 2.9% incorrect labels. The worst case was one of the normal subjects with 15 correctly-assigned labels vs. 4 incorrectly-assigned labels.

Airway Measurements: The automated airway measurements for the inner airway wall were validated with the help of a PLEXIGLAS airway phantom containing tubes with diameters of 1.98 mm to 19.25 mm. The validation on a phantom was acceptable, but not ideal—an in-vivo gold standard is preferred. Unfortunately, such a standard is not available because it is impossible to take sub-millimeter reference measurements on a living organism. Table 3 lists the results of the error measurements.

TABLE 3
Measurement errors for different tubes at multiple scan-angles φ.
Mean μ and standard deviation σ values are given for
individual scan angles as well as for individual nominal tube diameters.
Scanner voxel size = 0.391 × 0.391 × 0.6 mm³.

| | | (dₙₒₘ − d_{μ}) [mm] for Tube n | | | | | | | |
|---|---|---|---|---|---|---|---|---|---|
| φ | | 1 | 2 | 3 | 4 | 5 | 6 | μ | σ |
| 0° | | −0.00 | 0.20 | −0.03 | −0.14 | −0.16 | 0.04 | −0.02 | 0.17 |
| 5° | | 0.11 | 0.07 | −0.02 | −0.12 | −0.15 | 0.07 | −0.01 | 0.14 |
| 30° | | −0.07 | −0.17 | −0.12 | −0.22 | −0.26 | −0.14 | −0.16 | 0.09 |
| 90° | | −0.31 | 0.01 | −0.14 | −0.12 | −0.24 | −0.22 | −0.17 | 0.15 |
| | μ | −0.07 | 0.03 | −0.08 | −0.15 | −0.20 | −0.06 | | |
| | σ | 0.18 | 0.15 | 0.06 | 0.05 | 0.06 | 0.14 | | |

The absolute average deviation from the nominal diameter was never greater than 0.26 millimeters. Compared with the voxel dimension of 0.391×0.391×0.6 mm³, sub-voxel accuracy can be claimed for the average values. The same accuracy was achieved for all airway orientations, even for those airways situated within the scanner plane.

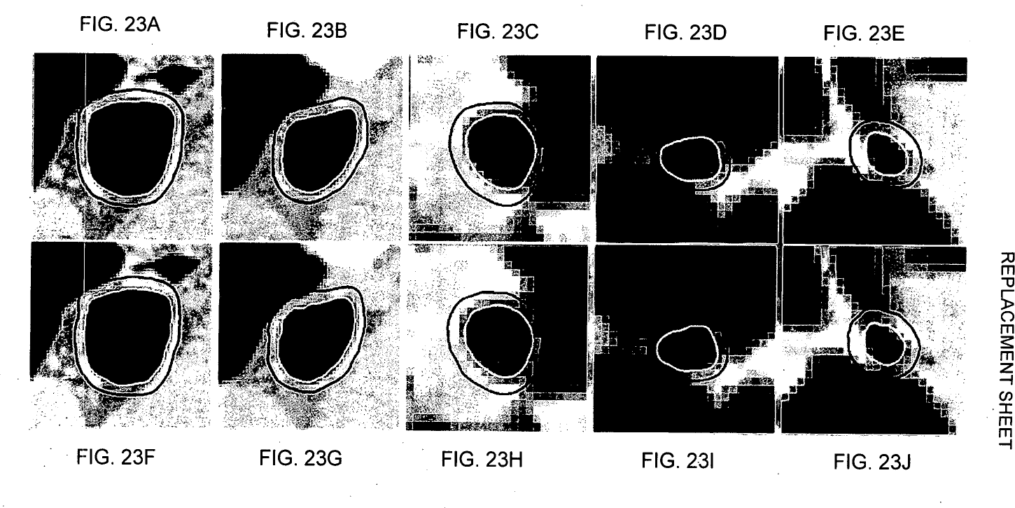

The segmentation of the outer airway wall was more realistically tested in in-vivo images because discontinuities caused by vessels are not represented in the PLEXIGLAS phantom that was available for validation. For that reason, a human expert hand-traced both the inner and the outer airway borders. The hand-drawn results were then compared with the automatically obtained borders. A qualitative comparison of the results (FIG. 23) shows a high degree of agreement.

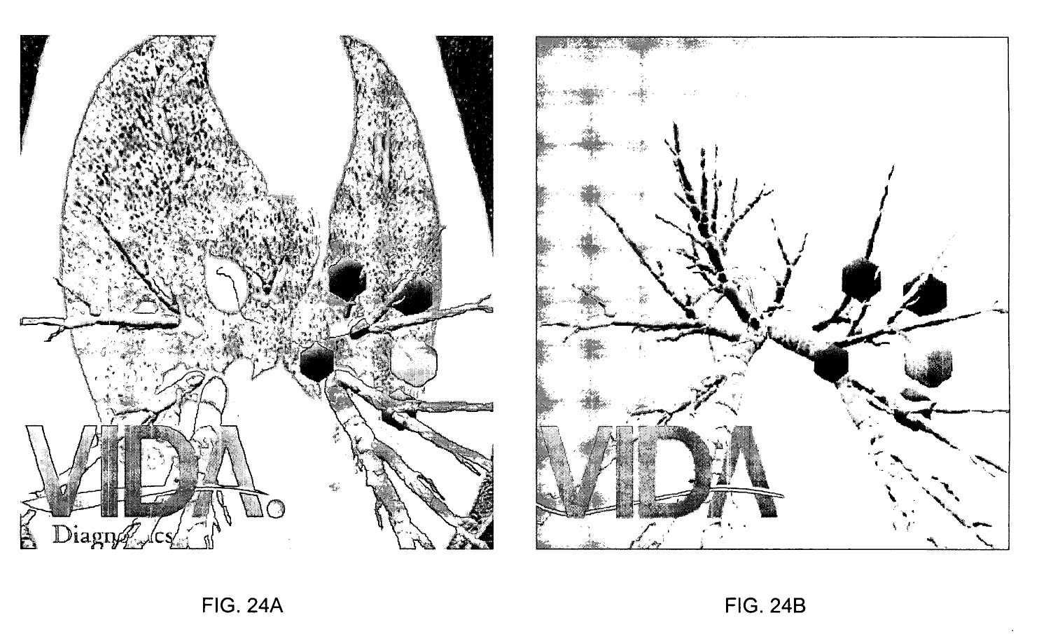

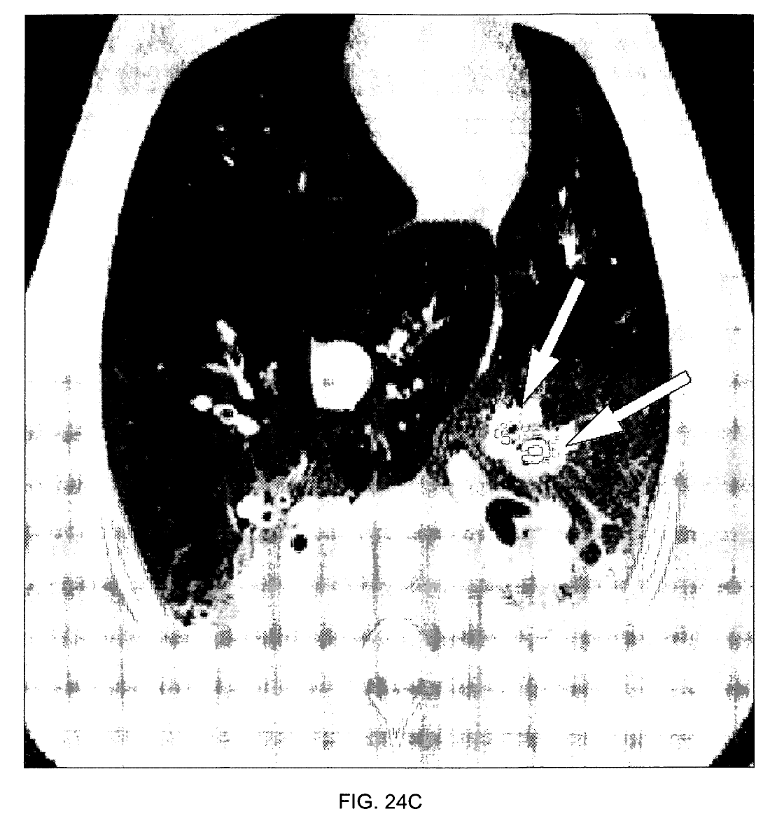

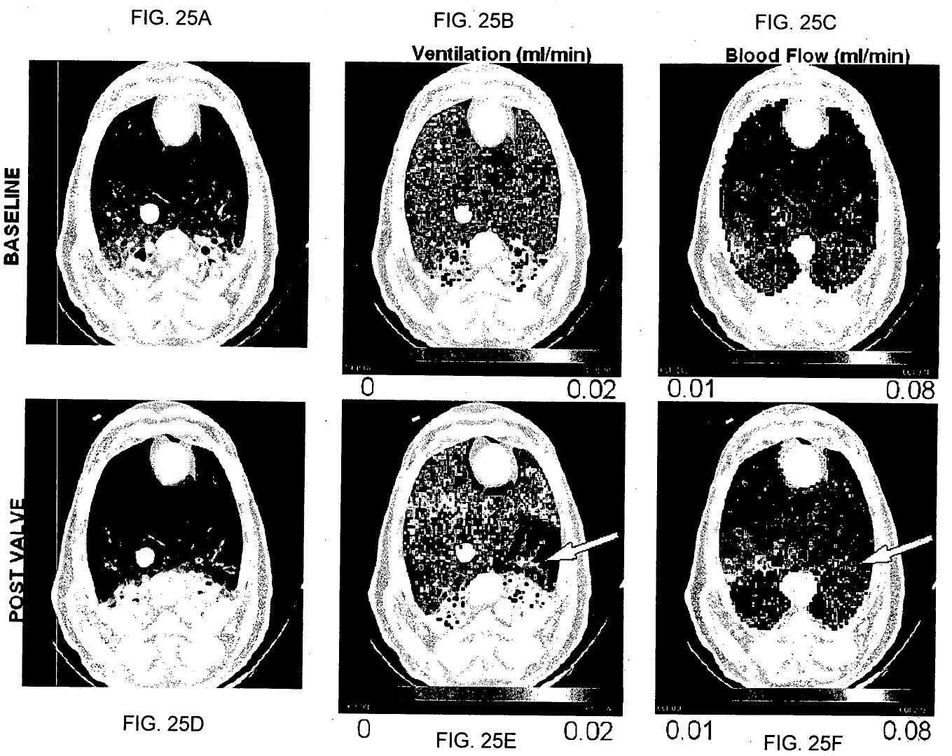

Planning valve placement and verifications: Sheep Experiment: Three sheep were placed supine in the I-CLIC Siemens Sensation 64 scanner, held apneic with air-way pressure of 25 cm H₂O during scanning. A thin slice (0.6 mm thick) spiral scan was obtained using 120 kV, 100 mAs. The Pulmonary Workstation (as described above) was used to segment the airway tree while simulated emphysema regions (identified by a technician) were shown as 3D spheres (see FIGS. 24A and 24B). By clicking on the region subtended by the spheres, the Pathfinder (as described above) provided the map through the airway tree. The bronchoscopist then placed two valves manufactured by Emphasys Medical Inc. (Redwood City, Calif.). The valves were sized based on the inner diameter of the target airway segment. Rather than waiting for a full collapse, Xenon CT (Tajik et al, 2002) and first pass blood flow methods were used to evaluate regional ventilation to the lung. The valves are shown as bright regions (see arrows in FIG. 24C) in the post placement scan. FIG. 25 depicts functional changes resulting from endobronchial valve placements, and shows the before (top row) and after (bottom row) ventilation (middle column) and perfusion (right column) images. Note the dramatic loss of ventilation (arrow shown in middle panel of FIG. 25) in the region targeted by the technician and followed by the bronchoscopist (red spheres in FIGS. 24A and 24B). Also note the reflex loss of blood flow in the same region shown in the lower right panel of FIG. 25.

Descriptions of well known processing techniques, components and equipment have been omitted so as not to unnecessarily obscure the present methods, devices and systems in unnecessary detail. The descriptions of the present methods, devices and systems are exemplary and non-limiting. Certain substitutions, modifications, additions and/or rearrangements falling within the scope of the claims, but not explicitly listed in this disclosure, may become apparent to those or ordinary skill in the art based on this disclosure.

For example, while this disclosure focuses on methods, devices and systems configured to help plan treatment for emphysema, there are wider applications for the present methods, devices and systems. For example, the functionality of the embodiment of Pathfinder discussed above may be used to assist a doctor in planning where to place a stent or some other type of patency-maintaining device, such as those disclosed in U.S. Patent Application Publication Nos. 2005/0137712 and 2005/0192526. As another example, the lobar segmentation techniques discussed above may be combined with lung nodule detection and characterization—which detection and characterization may be achieved either manually (e.g., a human operator uses a computer mouse to click on a suspected nodule), or automatically, using an algorithm known in the art—and used to plan surgical treatment for lung cancer. More specifically, the closest feeding airway to the area containing a lung nodule may be determined. The diameter and/or turn-radius characteristic(s) of a suitable therapeutic device—such as an radio frequency (RF) ablation catheter (and carrying bronchoscope), liquid nitrogen ablation device (and carrying bronchoscope) or radioactive bead insertion device—may also be determined. Additionally, certain dimensional parameters of the relevant portion of the airway tree may be determined. These data (e.g., in the form of inputs) may then be processed to identify (e.g., plot) a suitable (e.g., an optimal) route to the target treatment location.

As another example, the techniques discussed above in the Measurements of Airway Tree Geometry section that allow for the determination of certain airway tree measurements may be used to study airway reactivity and asthma, and to conduct longitudinal trials for asthma interventions, by comparing data on an anatomical basis (e.g., by lobe, named sub-lobar segment, named airway segment, and/or area distal to named airway segment). Such an anatomical-comparison technique may be important if a therapy is being measured that changes the geometry of the lung, such as pre- and post-asthma treatment comparison of airway wall thickness; direct measurement of lung volume distal to a lung valve or valves; and direct measurement of air trapping distal to an airway.

The appended claims are not to be interpreted as including means-plus-function limitations, unless such a limitation is explicitly recited in a given claim using the phrase(s) “means for” and/or “step for,” respectively.

### REFERENCES

The following references, to the extent that they provide exemplary procedural or other details supplementary to those set forth, are specifically incorporated by reference at the locations in this disclosure where they are respectively cited.
Alberle et al., J. Thorac. Imaging, 16(1):65-68, 2001.
Berlin, AJR Am. J. Roetgenol., 180(1):37-45, 2003.
Bolliger et al., Respiration, 71(1):83-87, 2004.
Borgefors, Computer Vision, Graphics, and Image Processing, 34(3):344-371, 1986.
Brown et al., IEEE Trans. Medical Imaging, 16:828-839, 1997.
Conforti and Shure, Pulmonary Perspectives, 17(4), 2000.
Ellis et al., Clin. Radiol., 56(9):691-699, 2001.
Ernst, Am. J. Respir. Crit. Care Med., 169(10):1081-1082, 2004.
Ferson and Chi, Thorac. Surg. Clin., 15(1):39-53, 2005.
Guo et al., In: Integrated system for CT-based assessment of parenchymal lung disease, Intl. Symposium on Biomed. Imaging, Washington, D.C., 871-874, 2002.
Hahn and Peitgen, Proc. SPIE Conf Medical Imaging, 5032:643-653, 2003.
Henschke and Yankelevitz, J. Thorac. Imaging, 15(1):21-27, 2000.
Henschke et al., Lancet., 354(9173):99-105, 1999.
Hu et al., IEEE Trans. Medical Imaging, 20:490-498, 2001.
Labadie et al., Curr. Opin. Otolaryngol. Head Neck Surg., 13(1):27-31, 2005.
Li et al., Proc. III Computer Soc. Conf Computer Vision Pattern Recogn., 1:394-399, 2004.
Li et al., Proc. SPIE Conf. Medical Imaging, 5370:620-627, 2004 (Li et al. II).
Lopez et al., Computer Vision and Image Understanding, 77:111-144, 2000.
Masutani et al., In: Region-growing based feature extraction algorithm for tree-like objects, Hohne and Kikinis (eds.), 1131:161-171, Springer, 1996.
Noppen et al., Chest, 125(2):723-730, 2004.
Palágyi et al, In: A sequential 3D thinning algorithm and its medical applications, 17^{th} Intl. Conf Inform. Proc. Med. Imaging, IPMI, 2082:409-415, 2001.
Reinhardt and Higgins, Optical Engineering, 37:570-581, 1998.
Russi and Weder, Swiss Med. Wkly, 132(39-40):557-561, 2002.
Sabanathan et al., et al., J. Cardiovasc. Surg., 44(1):101-108, 2003.
Silvela and Portillo, IEEE Trans. Image Proc., 10:1194-1199, 2001.
Sonka et al., In: Image Processing, Analysis and Machine Vision, PWS Publishing, 1999.
Swensen et al., Radiology, 235(1):259-265, 2005.
Tajik et al., Acad. Rad., 9(2):130-146, 2002.
Tschirren, In: Segmentation, anatomical labeling, branchpoint matching, and quantitative analysis of human airway trees in volumetric CT images, Thesis submitted for Doc. Of Phil., Elect. Comp. Eng., Univ. of Iowa, 2003.
Wu and Chen, In: Optimal net surface problems with applications, Proceed. of the 29^{th} Intl. Colloquium on Automata, Languages and Programming, Spain, 1029-1042, 2002.
Yim et al., J. Thorac. Cardiovasc. Surg., 127(6):1564-1573, 2004.

We claim:

1. A computer readable medium comprising machine readable instructions for:
determining a target treatment region within a lung;
providing a display of the target treatment region and an airway tree;
receiving a selection of a location for a therapeutic device within the airway tree; and
altering the display to indicate whether the location is therapeutically linked to the target treatment region.

2. The computer readable medium of claim 1, where the target treatment region is emphysematic.

3. The computer readable medium of claim 1, where the target treatment region is cancerous.

4. The computer readable medium of claim 1, where the target treatment region includes a lung nodule.

5. The computer readable medium of claim 1, where the selection comprises clicking on the location.

6. The computer readable medium of claim 1, where the altering comprises highlighting the target treatment region if the target treatment region is therapeutically linked to the location and not already highlighted.

7. A computer readable medium comprising machine readable instructions for:
displaying an airway tree and a target treatment region;
receiving a selection of a location for a therapeutic device within the airway tree; and
if the target treatment region is therapeutically linked to the location, indicating that the target treatment region is therapeutically linked to the location.

8. The computer readable medium of claim 7, where the target treatment region is emphysematic.

9. The computer readable medium of claim 7, where the target treatment region is cancerous.

10. The computer readable medium of claim 7, where the target treatment region includes a lung nodule.

11. The computer readable medium of claim 7, where the selection comprises clicking on the location.

12. The computer readable medium of claim 7, where the indicating comprises highlighting the target treatment region if the target treatment region is therapeutically linked to the location and not already highlighted.

13. A computer readable medium comprising machine readable instructions for:
displaying at least a portion of a set of lungs and one or more potential treatment regions;
receiving a selection of a target treatment region from among the one or more potential treatment regions; and
displaying one or more locations within the portion that are therapeutically linked to the target treatment region.

14. The computer readable medium of claim 13, where the target treatment region is emphysematic.

15. The computer readable medium of claim 13, where the target treatment region is cancerous.

16. The computer readable medium of claim 13, where the target treatment region includes a lung nodule.

17. The computer readable medium of claim 13, where the selection comprises clicking on one of the one or more potential treatment regions.

18. The computer readable medium of claim 13, where the displaying one or more locations comprises depicting a line extending from the target treatment region through at least a portion of an airway tree within the portion.

19. A computer readable medium comprising machine readable instructions for:
displaying at least a portion of a set of lungs and one or more potential treatment regions;
receiving a selection of a target treatment region from among the one or more potential treatment regions;
receiving an input of a location of a therapeutic device within the portion; and
if the target treatment region is therapeutically linked to the location, indicating that the target treatment region is therapeutically linked to the location.

20. The computer readable medium of claim 19, where the target treatment region is emphysematic.

21. The computer readable medium of claim 19, where the target treatment region is cancerous.

22. The computer readable medium of claim 19, where the target treatment region includes a lung nodule.

23. The computer readable medium of claim 19, where the selection comprises clicking on one of the one or more potential treatment regions.

24. The computer readable medium of claim 19, where the indicating comprises highlighting the target treatment region if the target treatment region is therapeutically linked to the location and not already highlighted.

25. A computer readable medium comprising machine readable instructions for:
displaying an airway tree segmented from a volumetric dataset of images;
receiving a selection of a portion of the airway tree; and
providing a display that includes the portion in straightened form and a dimensional attribute corresponding to a user-selectable location along the portion.

26. The computer readable medium of claim 25, where the portion is displayed in a lengthwise orientation, and the display also includes a second view of the portion that is oriented perpendicularly to the lengthwise orientation.

27. The computer readable medium of claim 26, where the display also includes a virtual view of the portion that is oriented perpendicularly to the lengthwise orientation.

28. The computer readable medium of claim 27, where the dimensional attribute has a value that can change in response to a user input, and the display also includes a location indicator that determines the value of the dimensional attribute.

29. The computer readable medium of claim 28, where the second view has a content that is determined by the location indicator.

30. The computer readable medium of claim 29, where the virtual view has a content that is determined by the location indicator.

31. The computer readable medium of claim 29, where the location indicator is superimposed on the lengthwise orientation of the portion.

32. An automated treatment planning method comprising:
determining a target treatment region within a lung;
providing a display of the target treatment region and an airway tree;
receiving a selection of a location for a therapeutic device within the airway tree; and
altering the display to indicate whether the location is therapeutically linked to the target treatment region.

33. The automated method of claim 32, where the target treatment region is emphysematic.

34. The automated method of claim 32, where the target treatment region is cancerous.

35. The automated method of claim 32, where the target treatment region includes a lung nodule.

36. The automated method of claim 32, where the selection comprises clicking on the location.

37. The automated method of claim 32, where the altering comprises highlighting the target treatment region if the target treatment region is therapeutically linked to the location and not already highlighted.

38. An automated treatment planning method comprising:
displaying an airway tree and a target treatment region;
receiving a selection of a location for a therapeutic device within the airway tree; and
if the target treatment region is therapeutically linked to the location, indicating that the target treatment region is therapeutically linked to the location.

39. The automated method of claim 38, where the target treatment region is emphysematic.

40. The automated method of claim 38, where the target treatment region is cancerous.

41. The automated method of claim 38, where the target treatment region includes a lung nodule.

42. The automated method of claim 38, where the selection comprises clicking on the location.

43. The automated method of claim 38, where the indicating comprises highlighting the target treatment region if the target treatment region is therapeutically linked to the location and not already highlighted.

44. An automated treatment planning method comprising:
displaying at least a portion of a set of lungs and one or more potential treatment regions;
receiving a selection of a target treatment region from among the one or more potential treatment regions; and
displaying one or more locations within the portion that are therapeutically linked to the target treatment region.

45. The automated method of claim 44, where the target treatment region is emphysematic.

46. The automated method of claim 44, where the target treatment region is cancerous.

47. The automated method of claim 44, where the target treatment region includes a lung nodule.

48. The automated method of claim 44, where the selection comprises clicking on one of the one or more potential treatment regions.

49. The automated method of claim 44, where the displaying one or more locations comprises depicting a line extending from the target treatment region through at least a portion of an airway tree within the portion.

50. An automated treatment planning method comprising:
displaying at least a portion of a set of lungs and one or more potential treatment regions;
receiving a selection of a target treatment region from among the one or more potential treatment regions;
receiving an input of a location of a therapeutic device within the portion; and
if the target treatment region is therapeutically linked to the location, indicating that the target treatment region is therapeutically linked to the location.

51. The automated method of claim 50, where the target treatment region is emphysematic.

52. The automated method of claim 50, where the target treatment region is cancerous.

53. The automated method of claim 50, where the target treatment region includes a lung nodule.

54. The automated method of claim 50, where the selection comprises clicking on one of the one or more potential treatment regions.

55. The automated method of claim 50, where the indicating comprises highlighting the target treatment region if the target treatment region is therapeutically linked to the location and not already highlighted.

56. An automated treatment planning method comprising:
displaying an airway tree segmented from a volumetric dataset of images;
receiving a selection of a portion of the airway tree; and
providing a display that includes the portion in straightened form and a dimensional attribute corresponding to a user-selectable location along the portion.

57. The automated method of claim 56, where the portion is displayed in a lengthwise orientation, and the display also includes a second view of the portion that is oriented perpendicularly to the lengthwise orientation.

58. The automated method of claim 57, where the display also includes a virtual view of the portion that is oriented perpendicularly to the lengthwise orientation.

59. The automated method of claim 58, where the dimensional attribute has a value that can change in response to a user input, and the display also includes a location indicator that determines the value of the dimensional attribute.

60. The automated method of claim 59, where the second view has a content that is determined by the location indicator.

61. The automated method of claim 60, where the virtual view has a content that is determined by the location indicator.

62. The automated method of claim 60, where the location indicator is superimposed on the lengthwise orientation of the portion.

63. A computer system configured to perform the automated methods of any of claims 32-62.
